(19) United States
(12) Patent Application Publication
(10) Pub. No.: US 2024/0350141 A1
(43) Pub. Date: Oct. 24, 2024

(54) BLOOD VESSEL CONNECTION SHUNT

(71) Applicant: EDWARDS LIFESCIENCES CORPORATION, Irvine, CA (US)

(72) Inventors: Zachary Christopher Berwick, Tustin, CA (US); Allen Jeong Keel, Irvine, CA (US); Yaeer E. Lev, Torrance, CA (US); Farzad Azimpour, Irvine, CA (US); Afnan Raouf Tariq, Newport Beach, CA (US)

(21) Appl. No.: 18/656,361

(22) Filed: May 6, 2024

Related U.S. Application Data
(63) Continuation of application No. PCT/US2022/048539, filed on Nov. 1, 2022.
(60) Provisional application No. 63/277,063, filed on Nov. 8, 2021.

(51) Int. Cl. A61B 17/11

(52) U.S. Cl. CPC A61B 17/11; A61B 2017/1107; A61B 2017/1135

(57) ABSTRACT

A shunt device comprises a first shunt portion configured to maintain a blood flow pathway between a first blood vessel and a second blood vessel, a first proximal flange configured to anchor the first shunt portion in place and configured for placement within the first blood vessel, and a first distal flange configured to anchor the first shunt portion in place. The first shunt portion is situated at least partially between the first proximal flange and the first distal flange.

### RELATED APPLICATION

This application is a continuation of International Patent Application No. PCT/US2022/048539, filed Nov. 1, 2022, and entitled BLOOD VESSEL CONNECTION SHUNT, which claims priority to U.S. Provisional Application No. 63/277,063, filed on Nov. 8, 2021, entitled BLOOD VESSEL CONNECTION SHUNT, the disclosures of which are hereby incorporated by reference in their entirety.

### BACKGROUND

The present invention relates generally to the field of medical devices and procedures.

Pulmonary hypertension is a rapidly deteriorating vascular disease associated with high short-term mortality rates. A primary driver of disease progression is the increase in pulmonary arterial pressure due to a reduction in vascular compliance.

### SUMMARY

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 illustrates an example representation of a heart 1 including indicators representing blood flow through the heart 1.

FIGS. 2A and 2B illustrate optional delivery methods for delivering one or more shunt implants in accordance with one or more examples.

FIGS. 3A and 3B illustrate an example shunt implant comprising two flanges in accordance with some examples.

FIGS. 4A and 4B illustrate another example shunt device configured to shunt blood between blood flow pathways and/or cardiac chambers in accordance with one or more examples.

FIG. 5 illustrates an example delivery system for a shunt device in accordance with one or more examples.

FIGS. 6A and 6B illustrate another example shunt device including flanges comprising one or more self-expanding arms configured to secure a shunt portion in place, in accordance with one or more examples.

FIGS. 7A and 7B illustrate an example shunt device delivered at least partially within and/or through one or more tissue walls via one or more catheters and/or other delivery systems in accordance with one or more examples.

FIGS. 8A and 8B illustrate another example shunt device delivered at least partially within and/or through two or more tissue walls via one or more catheters and/or other delivery systems in accordance with one or more examples

FIGS. 9A-9C illustrate delivery of an example shunt device at a tissue wall using various delivery systems.

FIGS. 10A-10C illustrate delivery steps of another example shunt device comprising one or more self-expanding and/or otherwise expandable anchoring arms.

FIGS. 11A and 11B illustrate another example shunt device deployed at least partially within a tissue wall in accordance with one or more examples.

FIGS. 12A and 12B illustrate delivery steps of another example shunt device comprising one or more self-expanding and/or otherwise expandable anchoring arms.

FIGS. 13A and 13B illustrate example shunt devices comprising one or more distal flanges configured to extend approximately perpendicularly from proximal flanges to facilitate delivery of the shunt devices across generally perpendicular blood vessels in accordance with one or more examples.

FIGS. 14A and 14B illustrate an intersection between a first blood vessel and a second blood vessel.

FIGS. 15A and 15B illustrate an example shunt device comprising a gasket and/or pressure-dependent orifice regulator that can modulate an amount of shunting to preferentially divert blood flow at higher pressures and minimize diversion at healthier pulmonary arterial pressures, in accordance with one or more examples.

FIGS. 16A-16C illustrate cross-sectional views of example deformable shunt devices, in accordance with one or more examples.

FIGS. 17A and 17B illustrate an example shunt device which may be configured for placement at a junction and/or intersection between two blood vessels in accordance with one or more examples.

FIGS. 18A-18C illustrate another example shunt implant configured to shunt blood between blood flow pathways and/or cardiac chambers in accordance with one or more examples

FIG. 19 illustrates a flowchart related to an example process for delivering one or more shunt implants to target locations within a body, which can include an intersection area between the RPA and the SVC, in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

### Overview

The following includes a general description of human cardiac anatomy that is relevant to certain inventive features and examples disclosed herein and is included to provide context for certain aspects of the present disclosure.

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

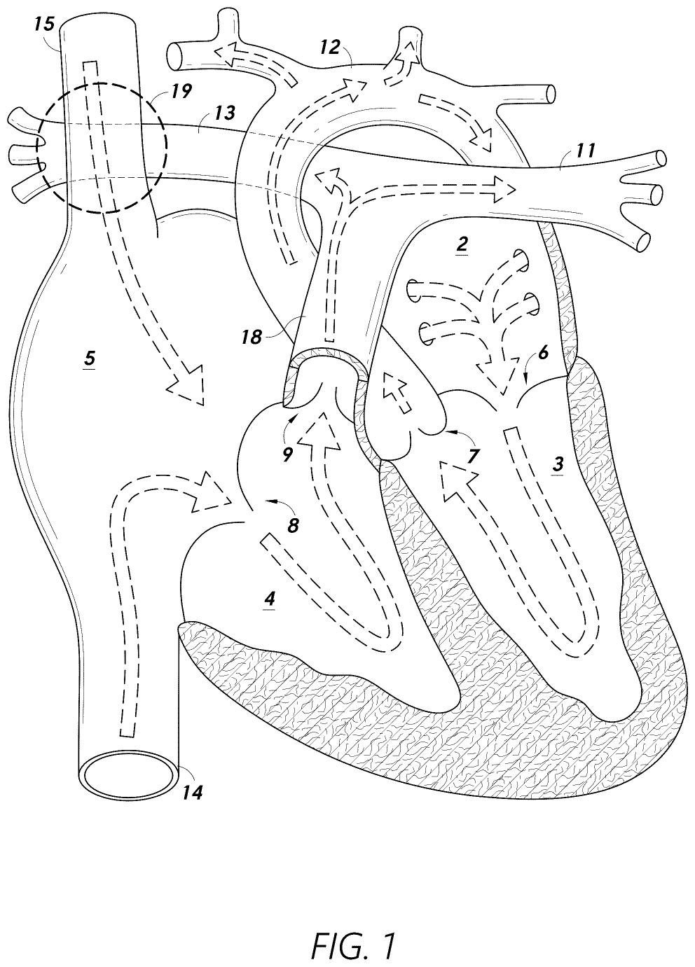

FIG. 1 illustrates an example representation of a heart 1 including indicators representing blood flow through the heart 1. The heart 1 includes four chambers, namely the left atrium 2, the left ventricle 3, the right ventricle 4, and the right atrium 5. A wall of muscle, referred to as the septum, separates the left 2 and right 5 atria and the left 3 and right 4 ventricles.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 18, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

Any of several access pathways may be utilized for maneuvering guidewires and catheters in and around the heart 1 to deploy medical implants (e.g., shunts) of the present application. For instance, access may be from above via either the subclavian vein or jugular vein into the superior vena cava (SVC) 15, right atrium (RA) 5 and from there into the coronary sinus ostium (CSO) 17. In another example, the access path may start in the femoral vein and through the inferior vena cava (IVC) 14 into the heart 1. Other access routes may also be used, and each typically utilizes a percutaneous incision through which the guidewire and catheter are inserted into the vasculature, normally through a sealed introducer, and from there the physician controls the distal ends of the devices from outside the body.

The pulmonary artery 18 branches into a right pulmonary artery (RPA) 13 and a left pulmonary artery (LPA) 11. Some examples of the present disclosure may involve delivering one or more implants (e.g., shunts) to an intersection 19 and/or crossing point between the RPA 13 and the SVC 15. For example, the RPA 13 may extend generally perpendicularly to the SVC 15 and/or can cross behind/in front of the SVC 15. In some cases, the RPA 13 may contact the SVC 15 while in other cases there may be a separation between the RPA 13 and the SVC 15.

Some examples described herein involve delivering a shunt system percutaneously to connect the RPA 13 with the SVC 15. Given that the RPA 13 and SVC 15 are adjacent anatomically, the intersection 19 area of the two provides an ideal location to establish a shunt. Further, because the RPA 13 has higher pressures than the SVC 15, particularly under pulmonary hypertensive conditions, unidirectional movement of blood flow is consistently diverted out of the RPA 13 and into the SVC 15. The net result of this shunting is to decompress and lower the pressure in the main pulmonary artery 18, including mean and peak systolic pressure. This is turn reduces the afterload on the right ventricle 4 and reduces the amount of work required to eject blood, thereby decreasing right ventricle 4 compensatory responses to pulmonary hypertension and preserving ventricular-vascular coupling. Shunting in some examples herein may occur at all pressures, or a shunt system may be pre-loaded to dynamically shunt at an offset pressure.

Pulmonary hypertension is a rapidly deteriorating vascular disease associated with high short-term mortality rates. A primary driver of disease progression is the increase in pulmonary arterial pressure due to a reduction in vascular compliance. The reduction in compliance is caused by several key factors, namely remodeling of the microcirculation or arteriosclerosis due to elevated pressures or systemic inflammation, respectively. The consequence of sustained and progressive increases in pulmonary arterial pressure is right ventricular-vascular uncoupling, whereby the right ventricle is no longer able to compensate for the increase in afterload which is typically accomplished by increases in stroke volume and contractility. Once this uncoupling occurs, the right ventricle begins to dilate with increases in filling pressures that can lead to tricuspid regurgitation and peripheral venous congestion. The combination of reduced forward flow and increased backwards transmission of pressure results in a reduction of transpulmonary perfusion, loss of gas exchange, hypoxemia, impaired LV filling, venous congestion of peripheral organs, and ultimately cardiac failure. This invention seeks to reduce the pulmonary artery pressure (both mean pressure and systolic) which is associated with an increase in afterload and work performed by the right ventricle. Via reductions in pulmonary arterial pressure, examples of the present disclosure can preserve right ventricular function, attenuate the progressive remodeling that occurs, and/or prevent peripheral venous congestion and symptoms associated with poor transpulmonary perfusion. Some examples may be applicable across multiple types of pulmonary hypertensive conditions.

Examples of the present disclosure relate to various percutaneous shunting methods and/or shunting devices that may be delivered percutaneously to connect different blood flow pathways. While the disclosure herein focuses on connecting and/or shunting between the RPA 13 and the SVC 15, this is for illustrative purposes and the examples described herein can be applied to other areas of anatomy. Because the RPA 13 and SVC 15 are adjacent anatomically, the intersection point of the RPA 13 and the SVC 15 can provide an effective location to establish one or more shunts. Further, because the RPA 13 has higher pressures than the SVC 15, particularly under pulmonary hypertensive conditions, unidirectional movement of blood flow is consistently diverted out of the RPA 13 and into the SVC 15. The net result of this shunting is to decompress and lower the pressure in the main pulmonary artery 18, including mean and peak systolic pressure. This is turn reduces the afterload on the right ventricle 4 and reduces the amount of work required to eject blood, thereby decreasing right ventricle 4 compensatory responses to pulmonary hypertension and preserving ventricular-vascular coupling. The various shunting methods described herein may be performed at any pressures and/or the various shunt devices described herein may be pre-loaded to dynamically shunt at an offset pressure.

The present disclosure provides methods and devices (including various medical implants) for shunting blood within a human body. The term “implant” is used herein according to its plain and/ordinary meaning and may refer to any medical implant, frame, valve, shunt, stent, anchor, and/or similar devices for use in treating various conditions in a human body. Implants may be delivered via catheter (i.e., transcatheter) for various medical procedures and may have a generally sturdy and/or flexible structure. The term “catheter” is used herein according to its broad and/ordinary meaning and may include any tube, sheath, steerable sheath, steerable catheters, and/or any other type of elongate tubular delivery device comprising an inner lumen configured to slidably receive instrumentation, such as for positioning within an atrium or coronary sinus, including for example delivery catheters and/or cannulas.

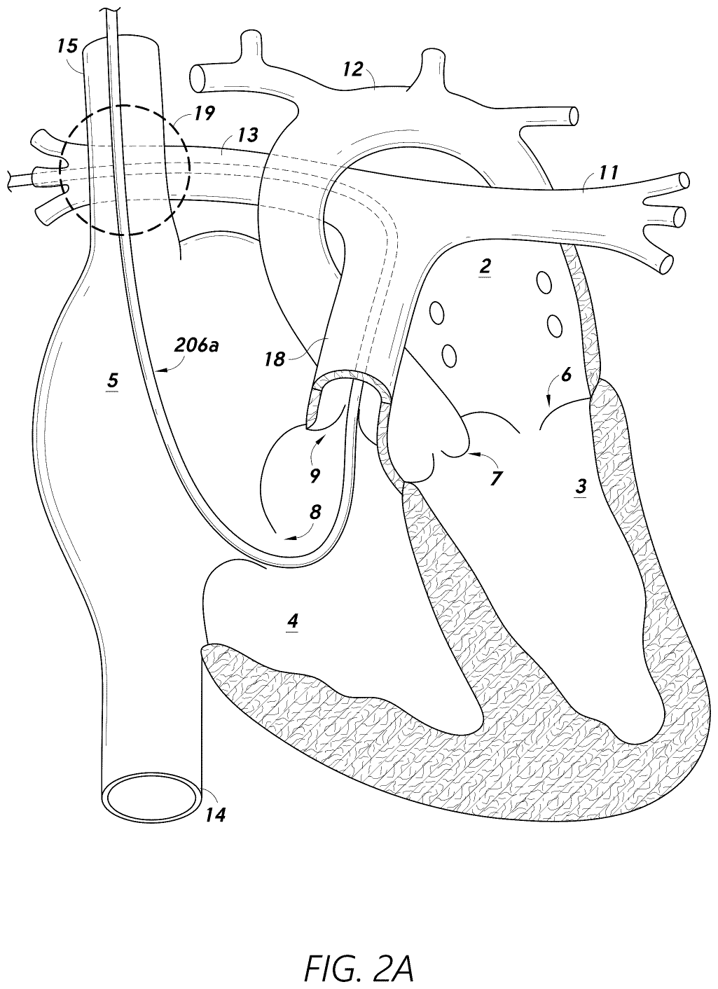

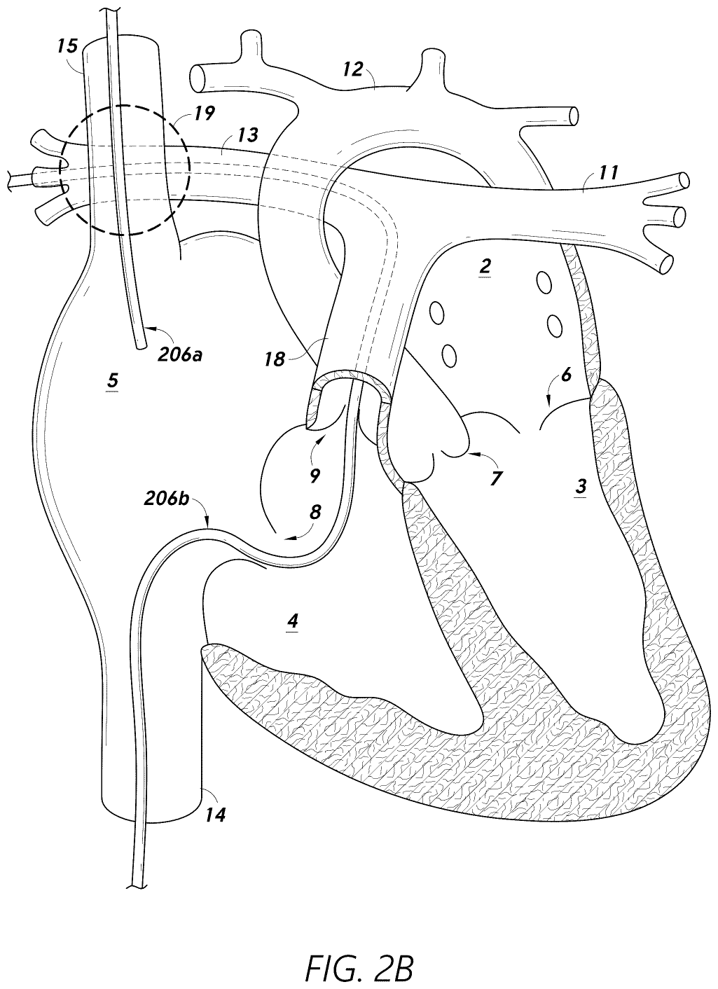

FIGS. 2A and 2B illustrate optional delivery methods for delivering one or more implants described herein. Some transcatheter processes described herein can utilize a single catheter 206 or multiple catheters 206. For example, FIG. 2A illustrates an example in which a first catheter 206a may be used to deliver one or more implants to the SVC 15 and/or the RPA 13. The first catheter 206a may be delivered to the SVC 15 and/or RPA 13 via any suitable delivery path. For example, the first catheter 206a may be delivered via the RPA 13 and the PA 18 and through the pulmonary valve 9 into the right ventricle 4, through the tricuspid valve 8 into the right atrium 5, and from the right atrium 5 into the SVC 15. Additionally or alternatively, the first catheter 206a may be delivered via the SVC 15 into the right atrium 5, through the tricuspid valve 8 into the right ventricle 4, through the pulmonary valve 9 into the PA 18 and finally into the RPA 13. Regardless of which delivery path is used, one or more implants may be delivered at the RPA 13 and/or SVC 15 at or near the intersection area 19.

FIG. 2B illustrates another example in which two catheters, a first catheter 206a and a second catheter 206b are delivered and/or used simultaneously. The first catheter 206a may be delivered via the SVC 15 and/or into the right atrium 5. The second catheter 206b may be delivered via the RPA 13 and the PA 18 and through the pulmonary valve 9 into the right ventricle 4, through the tricuspid valve 8 into the right atrium 5, and from the right atrium 5 out the IVC 14. Additionally or alternatively, the catheter 206 may be delivered via the IVC 14 into the right atrium 5, through the tricuspid valve 8 into the right ventricle 4, through the pulmonary valve 9 into the PA 18 and finally into the RPA 13. Regardless of which delivery path is used, one or more implants may be delivered at the RPA 13 and/or SVC 15 at or near the intersection area 19.

The one or more catheters 206 can be delivered via a transjugular, brachial, subclavian, and/or transfemoral approach. Within each access point, either one or two catheters 206 may be used for the delivery. Where two catheters 206 are used, the first catheter 206a may comprise a stent snare catheter and/or the second catheter 206b can comprise a puncture delivery catheter, or vice versa. In the case of a single catheter system, a puncture may be made and/or one or more implants delivered in one direction using the same catheter 206, in either SVC 15 to RPA 13 or vice versa directions. In some cases, it can be easier to deliver a catheter 206 through the RPA 13 than the SVC 15 and/or IVC 14.

The intersection area 19 between the RPA 13 and the SVC 15 may provide a suitable and/or desirable shunting location due at least in part to proximity and/or contact between the RPA 13 and SVC 15. In some cases, it may be advantageous to shunt from the RPA 13 to the SVC 15, particularly for patients experiencing pulmonary hypertension. For example, patients experiencing pulmonary hypertension can experience increases in pressure that can support the benefit of an inter-vasculature shunt. In some cases, the amount of disease in the pulmonary circulation can become independent of the left atrium. For example, even as left atrium pressure increases, pulmonary pressures can increase disproportionately to the left atrium pressure. As another example, even when left atrium pressure stabilizes, pulmonary pressure can continue to increase. Thus, an inter-vasculature shunt in a patient with pulmonary hypertension may provide incremental improvement, for example in comparison to unloading the pressure directly from the pulmonary circulation. By offloading pressure, right ventricle function can be improved. When the right ventricle starts to deteriorate, survival rate can decrease quickly. Any decrease in pressure, either locally or otherwise, can significantly reduce the amount of work the right ventricle has to perform to continue to pump blood to the left side of the heart.

The right side of heart (e.g., the right ventricle) may not be able to support relatively high loads. In contrast, the left side of the heart can be more suited to supporting large fluctuations of volume into itself. Thus, it can be beneficial to relieve pressure from the right side of the heart and instead moderately increase and/or redirect pressure to the left side.

A direction of flow between the RPA 13 and SVC 15 can be determined naturally. For example, a patient's condition (e.g., pulmonary hypertension) can cause the pressure in the RPA 13 to be higher, thus directing flow from the RPA 13 to the SVC 15.

In some cases, it may be advantageous to insert one or more delivery systems from the RPA 13 into the right ventricle 4 to allow for wiring in the direction of more length. This may be particularly advantageous in RPA 13/SVC 15 shunting because of the perpendicular orientation of the RPA 13 and SVC 15. One or more delivery systems may be maintained in the right ventricle 4 to provide trackability and support for other delivery system.

While certain example delivery methods are illustrated in FIGS. 2A and 2B, other delivery methods may be used for the example devices described herein. For example, a single catheter 206 may be delivered via the SVC 15 and/or the IVC 14. In another example, a first catheter 206a and/or a second catheter 206b may be delivered via the RPA 13 to the SVC 15 to allow one or more shunt devices to be delivered from the SVC 15 to the RPA 13. Additionally or alternatively, a first catheter 206a and/or a second catheter 206b may be delivered via the SVC 15 to the RPA 13 to allow one or more shunt devices to be delivered from the RPA 13 to the SVC 15.

### Connection Shunt Implants

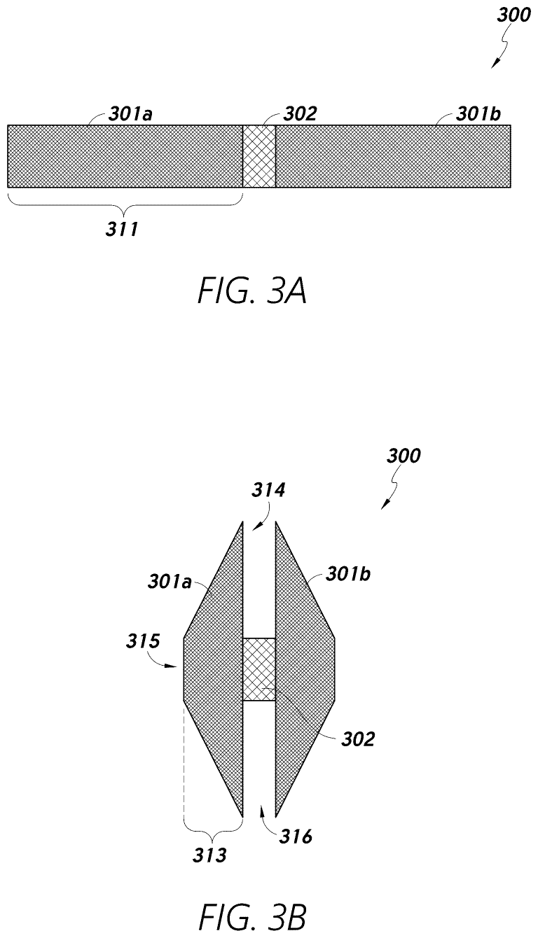

FIGS. 3A and 3B illustrate a shunt implant 300 comprising two flanges 301 in accordance with some examples. FIG. 3A illustrates a collapsed and/or compressed form of the implant 300, which the implant 300 may assume while within a catheter and/or other delivery system(s). The implant 300 may comprise any of a variety of features and/or components. In some examples, the implant 300 may be configured to form a connection and/or bridge between two or more blood vessels and/or chambers. The implant 300 may comprise a shunt portion 302 which may be configured to be situated between two or more tissue walls and/or at least partially within at least one tissue wall. The shunt portion 302 may be configured to create and/or maintain a blood flow pathway between and/or through the tissue walls. In some examples, the implant 300 may comprise multiple separate components which may be attached, connected, and/or otherwise joined to form a single device. For example, the shunt portion 302 may be coupled to one or more anchoring mechanisms 301 (e.g., flanges), which can include a distal anchoring mechanism 301a and/or a proximal anchoring mechanism 301b. The shunt portion 302 may form a generally tubular shape that can have a set/pre-formed size and/or a variable size.

The shunt portion 302 and/or anchoring mechanisms 301 can be at least partially composed of any suitable material(s), which can include expandable stainless steel, cobalt chromium, textiles, and/or Nitinol. In some examples, the shunt portion 302 and/or anchoring mechanisms 301 can be expanded via coaxial displacement of a delivery system (e.g., a catheter). The shunt effective orifice area (EOA) and/or diameter of the shunt portion 302 may be configured to support any desired amount of shunting. For example, the shunt portion 302 may be configured to achieve a minimum reduction in pulmonary pressure while preserving a transpulmonary pressure gradient required to facilitate pulmonary perfusion and delivery of blood to the left atrium. The shunt EOA and/or length of the shunt portion 302 may be configured to maintain a pressure reduction across a variety of clinical conditions, including but not limited to peripheral venous hypertension and exercise.

In some examples, the shunt portion 302 and/or anchoring mechanisms 301 can comprise a plurality of struts arranged longitudinally and/or circumferentially with varying thicknesses and/or mechanical properties. These variations in strut thickness can be situated to facilitate expansion of the shunt portion 302 when, for example, an inner catheter body is moved with respect to an outer catheter body.

In some examples, the shunt portion 302 and/or anchoring mechanisms 301 may be at least partially composed of braided materials, which may include stainless steel, Nitinol, and/or other metals, polymers, and/or textile materials, including flexible and/or braided textiles. Textile materials can include memory-formed textiles. At least a portion of the implant 300 may be configured to collapse to a smaller diameter for delivery while maintaining flexibility of the implant 300. In some examples, at least a portion of the implant 300 may be covered by a tubular sheath (not shown) configured to surround at least a portion of the implant 300 and/or the implant 300 may comprise a solid tubular material. The sheath may be configured to prevent the implant 300 from expanding from a crimped configuration. In some examples, additional and/or alternative devices and/or methods may be used to prevent expansion of the implant 300. For example, one or more wires may be attached to the implant 300 to prevent expansion of the implant prior to delivery.

The shunt portion 302 may be situated at least partially between the first anchoring mechanism 301a and the second anchoring mechanism 301b. The shunt portion 302, first anchoring mechanism 301a, and second anchoring mechanism 301b may form a channel and/or lumen through which blood can flow.

FIG. 3B illustrates an expanded and/or default configuration of the implant 300, which the implant 300 may assume following removal from a catheter and/or other delivery system(s).

As shown in FIG. 3B, the one or more anchoring mechanisms 301 may be configured to reduce in length during expansion. For example, the one or more anchoring mechanisms 301 may be at least partially composed of braided and/or interwoven cords, textiles, and/or similar devices that may be configured to bend, flex, and/or otherwise adjust to allow the anchoring mechanisms 301 to form varying shapes and/or sizes.

While the anchoring mechanisms 301 are shown having a conical and/or trapezoidal shape, the anchoring mechanisms 301 may have any suitable shape and/or form. In some examples, a diameter and/or width of an anchoring mechanism 301 may be variable and/or may increase to a maximal width and/or diameter at one or more proximal portions 314 configured to be situated in contact with and/or near a tissue wall and/or the shunt portion 302. Similarly, the width and/or diameter of the anchoring mechanism 301 may decrease to a minimal diameter and/or width at one or more distal portions 315 of the anchoring mechanisms 301.

FIGS. 3A and 3B provide side views of the shunt device 300. The shunt device 300 may have a generally tubular form and/or may have a generally circular cross section. In the compressed form shown in FIG. 3A, the one or more anchoring mechanisms 301 may have a first length 311, which may be a maximal length of the one or more anchoring mechanisms 301. As the shunt device 300 expands to the expanded form shown in FIG. 3B, the one or more anchoring mechanisms may naturally decrease in length to a second length 313, which may be a minimal length of the one or more anchoring mechanisms 301. Moreover, as the shunt device 300 expands, a lumen extending through the one or more anchoring mechanisms 301 and/or shunt portion 302 may decrease in diameter and/or width. The shunt device 300 may be configured to assume the expanded form shown in FIG. 3B upon and/or in response to removal from a catheter and/or other delivery device(s).

The one or more anchoring mechanisms 301 may be configured to expand around either side of a gap 316 between the anchoring mechanisms. In some examples, the one or more anchoring mechanisms 301 may be configured to sandwich and/or be positioned on either side of one or more tissue walls. For example, one or more tissue walls may be configured to fit within the gap 316 formed by the anchoring mechanisms 301. The one or more anchoring mechanisms 301 may be configured to form a hermetic seal around a tissue wall. In some examples, one or more anchoring mechanisms 301 may comprise a coating and/or covering (e.g., a polymer and/or other material) to improve a seal created by the anchoring mechanisms 301.

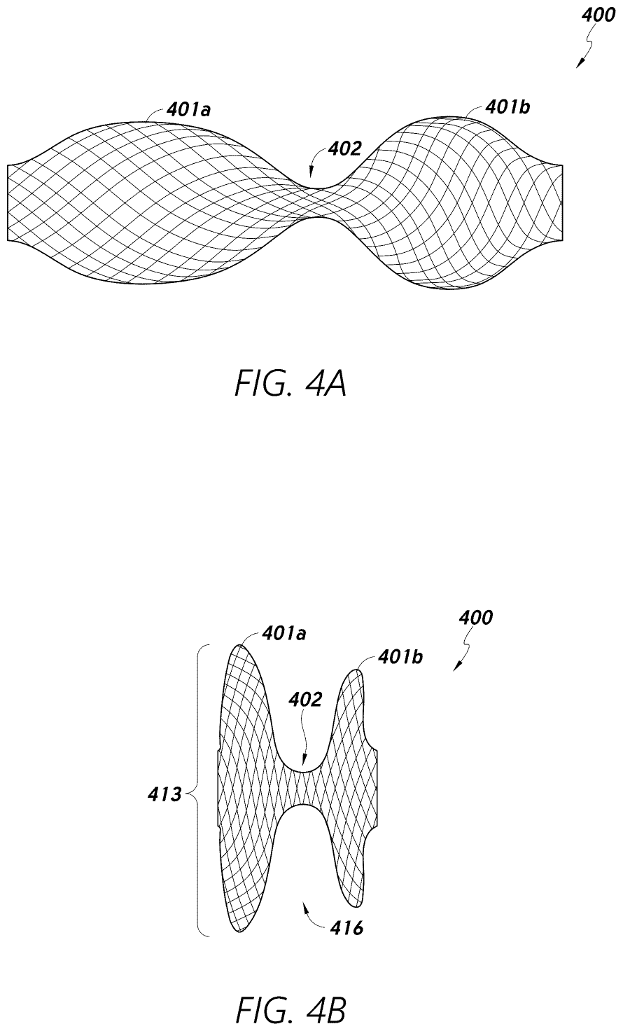

FIGS. 4A and 4B illustrate another example shunt device 400 configured to shunt blood between blood flow pathways and/or cardiac chambers. The shunt device 400 can include a shunt portion 402 situated between a first anchoring mechanism 401a and/or a second anchoring mechanism 401b. FIG. 4A illustrates an expanded form of the shunt device 400. For example, the shunt device 400 may be configured to naturally assume the form shown in FIG. 4A following removal from a catheter and/or other delivery systems. The shunt device 400 may be configured to collapse to a compressed form during delivery through one or more delivery systems. The first anchoring mechanism 401a and/or the second anchoring mechanism 401b may be configured to expand to a greater width and/or diameter than the shunt portion 402. In some examples, the shunt portion 402 may be configured to be positioned within one or more tissue walls and/or a space separating two blood flow pathways and/or chambers. The first anchoring mechanism 401a and/or the second anchoring mechanism 401b may be configured to secure the shunt portion 402 in place to maintain a blood flow pathway through the shunt portion 402.

As shown in FIG. 4B, the first anchoring mechanism 401a and/or the second anchoring mechanism 401b may be configured to collapse and/or compress lengthwise to decrease the lengths of the first anchoring mechanism 401a and/or second anchoring mechanism 401b and/or to increase the widths of the anchoring mechanisms 401 to a first width 413. In some examples, the first anchoring mechanism 401a and/or the second anchoring mechanism 401b may be configured to be pressed against one or more tissue walls. The anchoring mechanisms 401 may be configured to extend around a gap 416, which may allow one or more tissue walls to fit at least partially between the anchoring mechanisms 401.

The shunt portion 402, first anchoring mechanism 401a, and/or second anchoring mechanism 401b may be at least partially composed of one or more braided and/or interwoven materials to allow the shunt device 400 to be flexible and/or expandable. In some examples, the first anchoring mechanism 401a, the shunt portion 402, and/or the second anchoring mechanism 401b may comprise extensions of a singular device and/or may extend into each other.

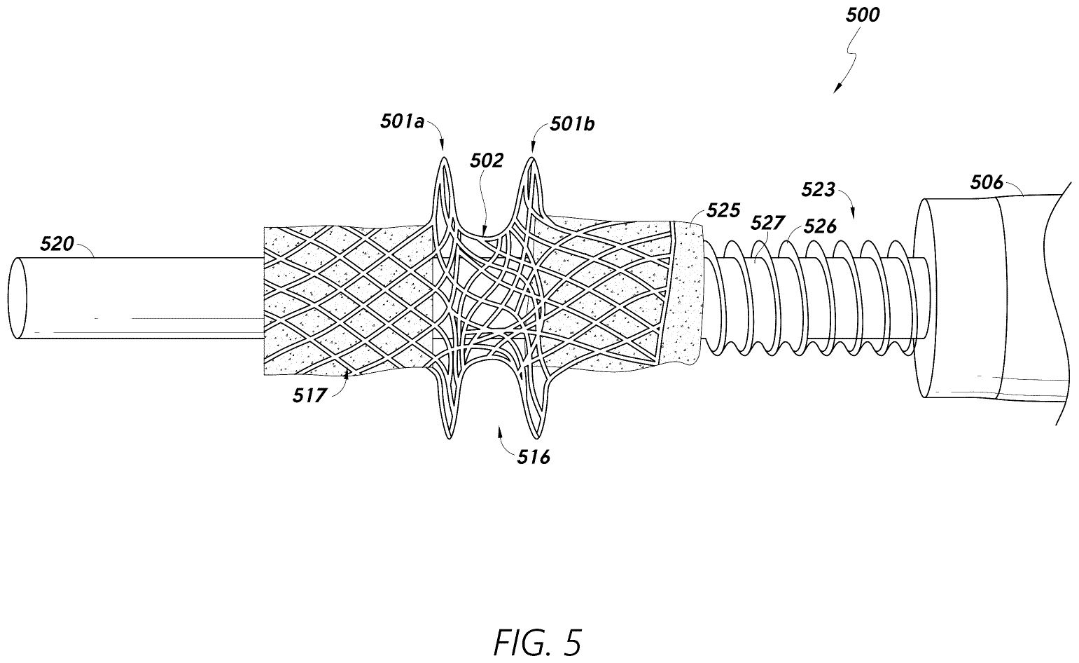

FIG. 5 illustrates an example delivery system 500 for a shunt device 517 in accordance with one or more examples. In some examples, compression and/or expansion of the shunt device 517 may be at least partially adjustable through use of a threaded wire member 523 configured to be twisted by an operator to press against one or more flanges 501 (e.g., a distal flange 501a and/or a proximal flange 501b) of the shunt device 517 and/or against one or more fixed portions 525 attached to the shunt device 517 to compress the one or more flanges lengthwise and/or expand the one or more flanges 501 in width. At least a portion of the shunt device may be crimped and/or otherwise coupled to one or more fixed portions 525 which may be configured to support the shunt device 517. As the threaded wire member 523 presses against the fixed portion 525 and/or the flanges 501, a length of the shunt device 517 may decrease and/or a width of the flanges 501 may increase. Similarly, a width and/or diameter of a lumen through the shunt device 517 may decrease as the threaded wire member 523 presses against the fixed portion 525 and/or flanges 501. The shunt device 517 may be adjusted until a desired amount of adjustment to pressure flow through the shunt device 517 and/or other therapeutic value is achieved.

The threaded wire member 523 may comprise one or more threads 526 (e.g., a single continuous thread) comprising one or more protrusions and/or teeth extending from a base member 527, which may have a generally tubular form. The base member 527 may extend into an inner catheter 520 and/or the inner catheter 520 may be separate and/or distinct from the base member 527. The inner catheter 520 and/or base member 527 may be configured to extend at least partially through the shunt device 517 and/or fixed portion 525. The shunt device 517, fixed portion 525, inner catheter 520, and/or threaded wire member 523 may be configured to fit at least partially within a catheter 506.

The shunt device 517 may comprise generally tubular portions, including a shunt portion 502, and/or one or more flanges 501. The flanges 501 may be configured to extend around a gap 516 to allow one or more tissue walls to fit at least partially between the flanges 501.

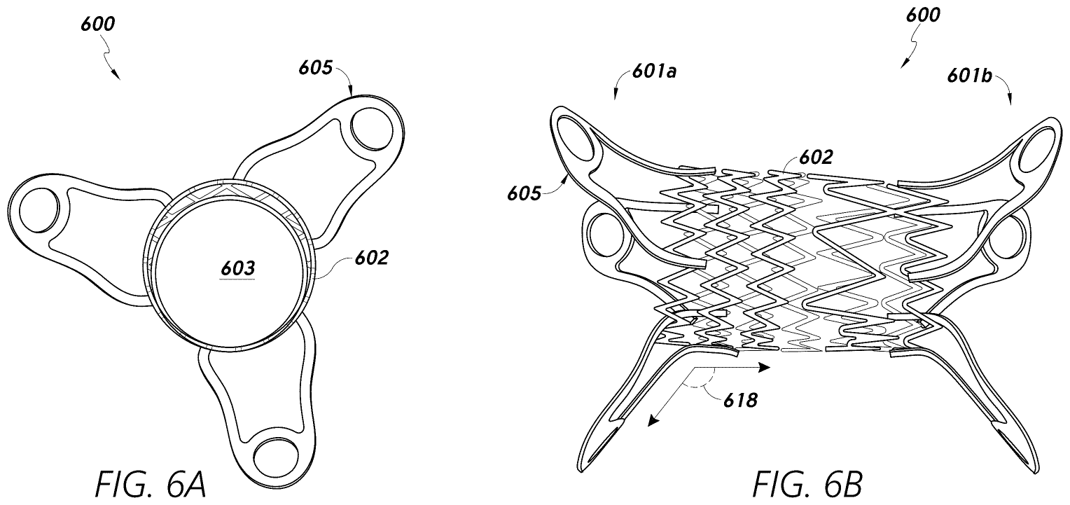

FIGS. 6A and 6B illustrate another example shunt device 600 including flanges 601 comprising one or more self-expanding arms 605 configured to secure a shunt portion 602 in place, in accordance with one or more examples. The shunt device 600 may represent a hybrid structure in relation to other shunt devices described herein and/or may have a generally flexible and/or bendable structure. The shunt portion 602 may have a generally tubular form and/or may be configured to at least partially enclose a cylindrical inner lumen 603. While the shunt device 600 is shown comprising three arms 605 at either side of the shunt portion 602, the shunt device 600 may comprise any number of arms 605. In some examples, the shunt portion 602 and/or arms 605 may be at least partially composed of a mesh and/or network of cords. The arms 605 may be configured to extend radially outward and/or generally perpendicularly to an approximately 70-110-degree angle 618 with respect to the shunt portion 602. In some examples, the arms 605 may be configured naturally expand and/or extend to the form shown in FIGS. 6A and 6B. The angle 618 may be variable and/or may be configured to naturally adjust to accommodate position and/or stability across a range of tissue wall thicknesses of blood vessels and/or chamber walls.

The shunt device 600 may comprise a distal flange 601a comprising a first set of one or more arms 605 and/or the shunt device 600 may comprise a proximal flange 601b comprising a second set of one or more arms 605. The shunt portion 602 may comprise a bare and/or covered network of cords and/or wires (e.g., metallic wires). The shunt portion 602 may have a generally flexible form and/or may be configured to allow for different degrees of flexibility and/or torsion. For example, the shunt portion 602 may have an adjustable length. For example, the shunt portion 602 may be at least partially composed of wires and/or similar devices configured to bend and/or stretch in response to external force. Thus, the shunt portion 602 and/or shunt device 600 may be adjustable to fit varying distances between vessels, which may be advantageous due at least in part to anatomic and/or pathologic variations.

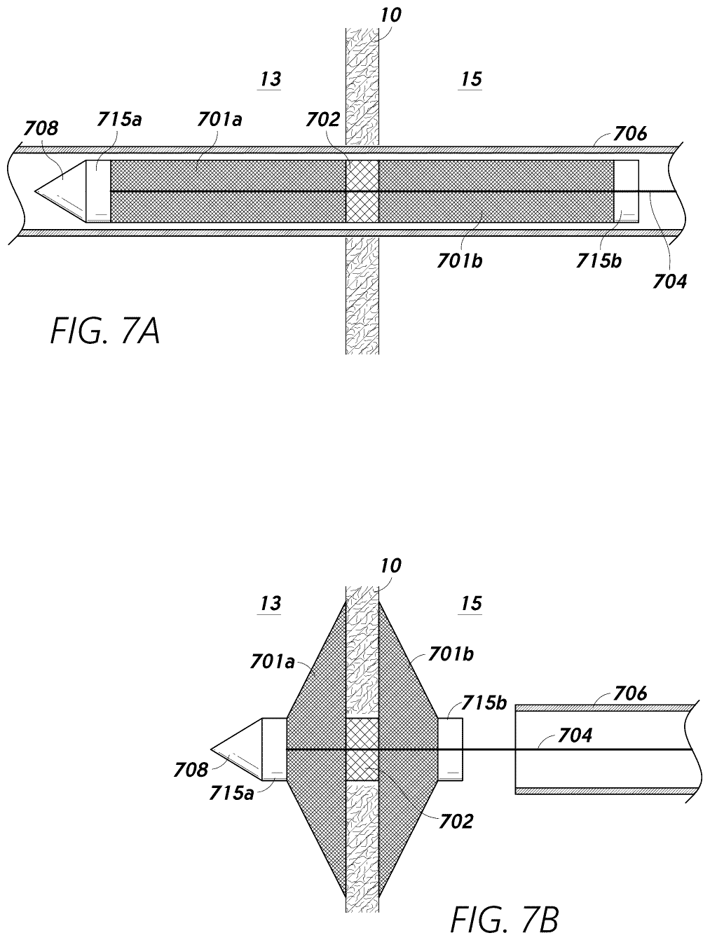

FIGS. 7A and 7B illustrate an example shunt device delivered at least partially within and/or through one or more tissue walls 10 via one or more catheters 706 and/or other delivery systems in accordance with one or more examples. A nose cone 708 having a generally conical and/or tapered form may be configured to lead at least a portion of the shunt device and/or to dilate and/or penetrate one or more tissue walls 10. In some examples, the tissue wall 10 may represent multiple tissue walls pressed together. For example, the tissue wall 10 may represent a tissue wall of an RPA 13 and/or a tissue wall of an SVC 15, which may be situated adjacent to each other. For adjacent anatomic structures, the shunt device can comprise only a single shunt portion 702 and/or two flanges 701 (e.g., expandable anchoring elements).

The delivery systems can comprise an inner rod 704 extending at least partially through the shunt device and/or coupling to a distal stopper 715a in contact with a distal flange 701a. The delivery systems can further comprise a proximal stopper 715b in contact with a proximal flange 701b. In some examples, the distal stopper 715a and the proximal stopper 715b can be drawn together and/or a distance between the distal stopper 715a and the proximal stopper 715b can otherwise be reduced to cause compression (e.g., lengthwise) and/or expansion (e.g., in width and/or diameter) of the flanges 701.

The flanges 701 can be deployed and/or expanded by withdrawing the inner rod 704 towards the operator, causing deformation and/or expansion of the flanges 701. The flanges 701 may be configured to continue to expand until distal portions of the flanges 701 (e.g., end of the flanges 701 that are distal from the tissue wall 10) come into contact and/or lock corresponding locking features.

In some examples, the shunt device can comprise one or more flared barbs on lumen-facing surfaces of the flanges 701 configured to stabilize the shunt device by anchoring the shunt device to the tissue wall 10. The distal flange 701 and/or proximal flange 701 may comprise one or more such flared barbs.

The shunt device may be at least partially composed of bare and/or enclosed metal and/or other material. In some examples, the shunt device may be bare in the case of adjacent anatomic structures (e.g., an adjacent RPA 13 and SVC 15) and/or the shunt device may be at least partially covered in the case of non-adjacent anatomic structures (e.g., where the shunt device comprises multiple shunt portions 702 and/or more than two flanges 701). For example, the at least partially covered shunt device may be configured to prevent infiltration of blood into the thoracic cavity and/or other anatomical area. In some examples, a diameter and/or width of the shunt device may be adjustable through approximation of the flanges 701 and/or stoppers 715.

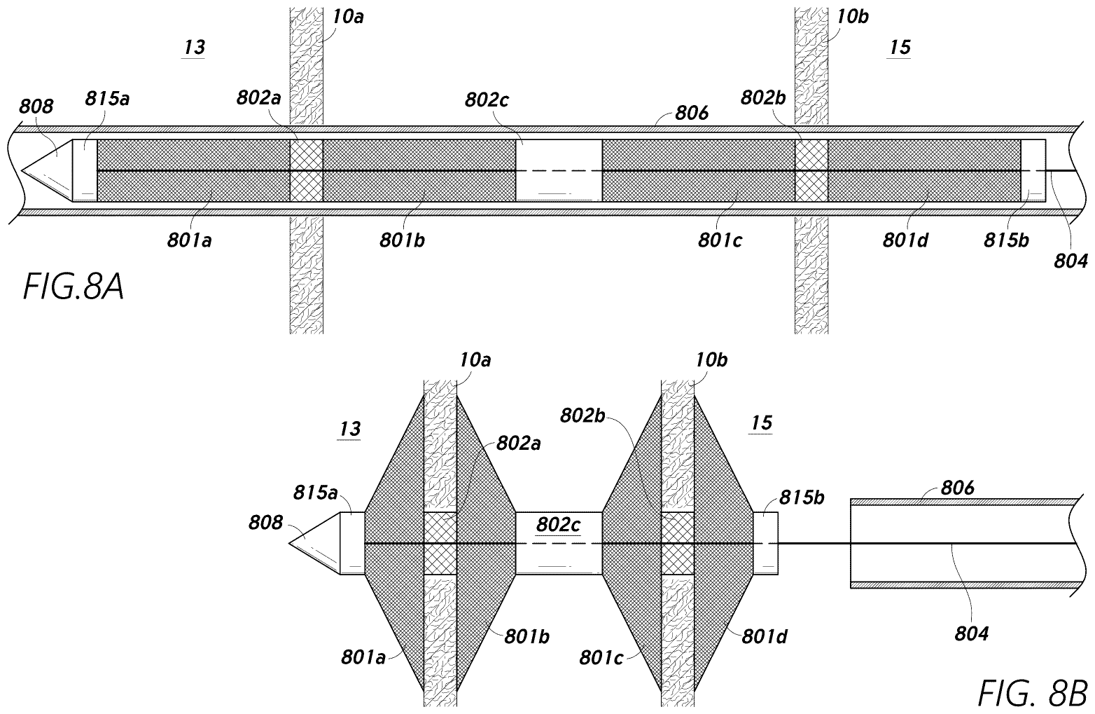

FIGS. 8A and 8B illustrate another example shunt device delivered at least partially within and/or through two or more tissue walls 10 via one or more catheters 806 and/or other delivery systems in accordance with one or more examples. The shunt device illustrated in FIGS. 8A and 8B may be utilized in cases of non-adjacent blood vessels and/or where there is a separation between the blood vessels. A first tissue wall 10a may be associated with a first blood vessel (e.g., an RPA) and/or a second tissue wall 10b may be associated with a second blood vessel (e.g., an SVC). For cases in which multiple blood vessels and/or tissue walls 10 are not adjacent and/or in contact, the shunt device can comprise more than two (e.g., four) expandable and/or compressible flanges 801, which can include two flanges 801 for each tissue wall 10 to effectively stabilize and/or prevent dislodgement of the shunt device. For example, the shunt device may comprise a first distal flange 801a and a second distal flange 801b configured to anchor to a distal tissue wall 10a and/or a first proximal flange 801c and/or a second proximal flange 801d configured to anchor to a proximal tissue wall 10b. The first distal flange 801a and the second distal flange 801b may be attached to a distal shunt portion 802a configured to shunt blood through the distal tissue wall 10a. The first proximal flange 801c and the second proximal flange 801d may be attached to a proximal shunt portion 802b configured to shunt blood through the proximal tissue wall 10b. The shunt device may further comprise a middle shunt portion 802c configured to connect and/or bridge the second distal flange 801b and the first proximal flange 801c.

In some examples, the one or more flanges 801 may be configured to sandwich and/or be positioned on either side of the one or more tissue walls 10. The one or more flanges 801 may be configured to form a hermetic seal around one or more tissue walls 10. In some examples, the flanges 801 can be configured to adjust distances between multiple blood vessels and/or tissue walls 10 of the blood vessels as a way of titrating the cross-sectional area of the shunt device. The distance between the tissue walls 10 can be adjusted until a target drop in pressure is achieved. In some examples, one or more flanges 801 may comprise a coating and/or covering to improve a seal created by the flanges 801.

In some examples, the various flanges 801 and/or shunt portions 802 of the shunt device may be configured to be moved and/or adjusted independently of each other. For example, a lumen of a distal shunt portion 802a may be adjusted independently of a lumen of the proximal shunt portion 802b and/or the middle shunt portion 802c. Independent adjustment of the shunt portions 802 and/or flanges 801 may be facilitated by separation between the various components and/or by holding one or more components stationary while adjusting others. For example, one or more collars and/or similar mechanisms may be used to crimp down between components of the shunt device. In another example, control wires may be used to selectively adjust and/or hold stationary one or more components.

The shunt device can comprise a distal shunt portion 802a, a proximal shunt portion 802b, and/or a middle shunt portion 802c. The distal shunt portion 802a may be configured to be situated at least partially within a distal tissue wall 10a and/or the proximal shunt portion 802b may be configured to be situated at least partially within a proximal tissue wall 10b. The middle shunt portion 802c may be configured to be situated in a space between the distal tissue wall 10a and the proximal tissue wall 10b. As the distal tissue wall 10a and proximal tissue wall 10b are pressed together, the tissue walls 10 may be configured to facilitate hermetic seals of the shunt device.

The shunt device may be configured for delivery via various delivery systems, which can include one or more catheters configured to deliver the shunt device and/or one or more guidewires 804, stoppers 815 (e.g., a distal stopper 815a and/or a proximal stopper 815b), and/or a nose cone 808.

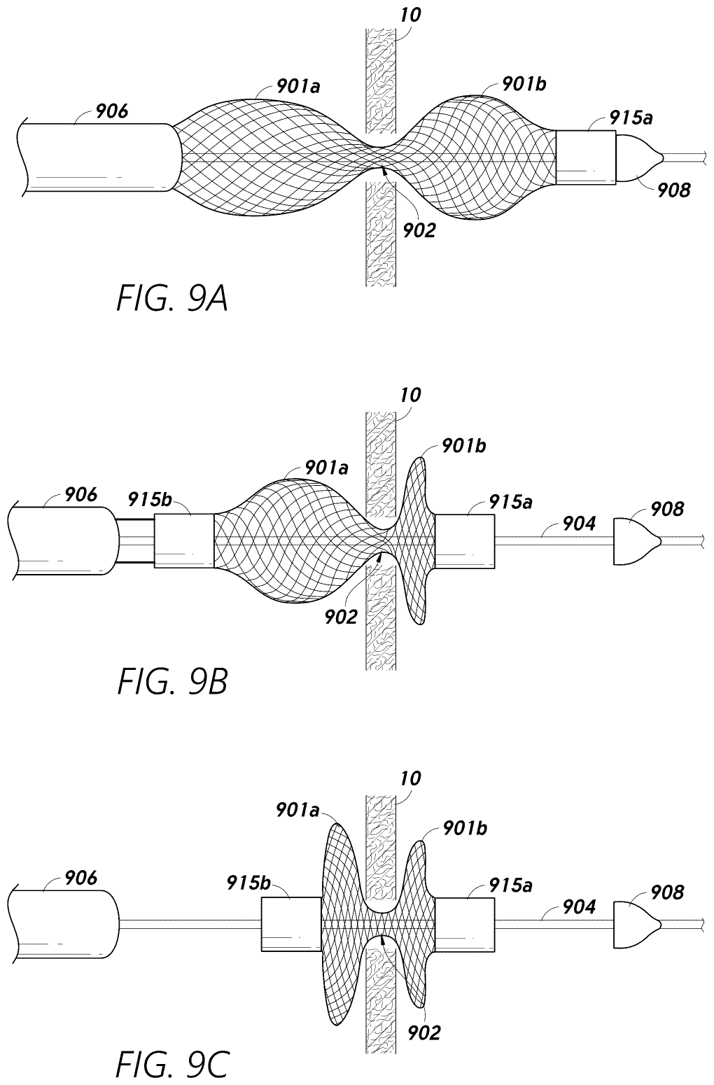

FIGS. 9A-9C illustrate delivery of an example shunt device at a tissue wall 10 using various delivery systems. FIG. 9A illustrates a first delivery stage in which the shunt device may have a default form. In the default form, one or more flanges 901 of the shunt device may have a generally expanded form. For example, the one or more flanges 901 may have generally spherical forms. FIG. 9B illustrates a second delivery stage of the shunt device in which a distal flange 901a is at least partially compressed using a distal stopper 915a. Compression of the distal flange 901a may cause a width and/or diameter of the distal flange 901a to increase and/or a lumen through the distal flange 901a and/or the shunt portion 902 to decrease in width and/or diameter. Moreover, compression of the distal flange 901a may cause the distal flange 901a to decrease in length. FIG. 9C illustrates a third delivery stage of the shunt device in which a proximal flange 901b is compressed using a proximal stopper 915b. Compression of the proximal flange 901b may cause a width and/or diameter of the proximal flange 901b to increase and/or a lumen through the proximal flange 901b and/or the shunt portion 902 to decrease in width and/or diameter. Moreover, compression of the proximal flange 901b may cause the proximal flange 901b to decrease in length. The tissue wall 10 may represent multiple tissue walls pressed together. In some examples, compression of the distal flange 901a and/or proximal flange 901b may cause multiple tissue walls to be pressed closer together. A nose cone 908 may be configured to facilitate delivery of the shunt device.

In some examples, the shunt device may be configured for delivery via a catheter 906 and/or other delivery device. A delivery system may further comprise a guidewire 904 and/or shaft configured to extend at least partially through the shunt device and/or to guide delivery of the shunt device.

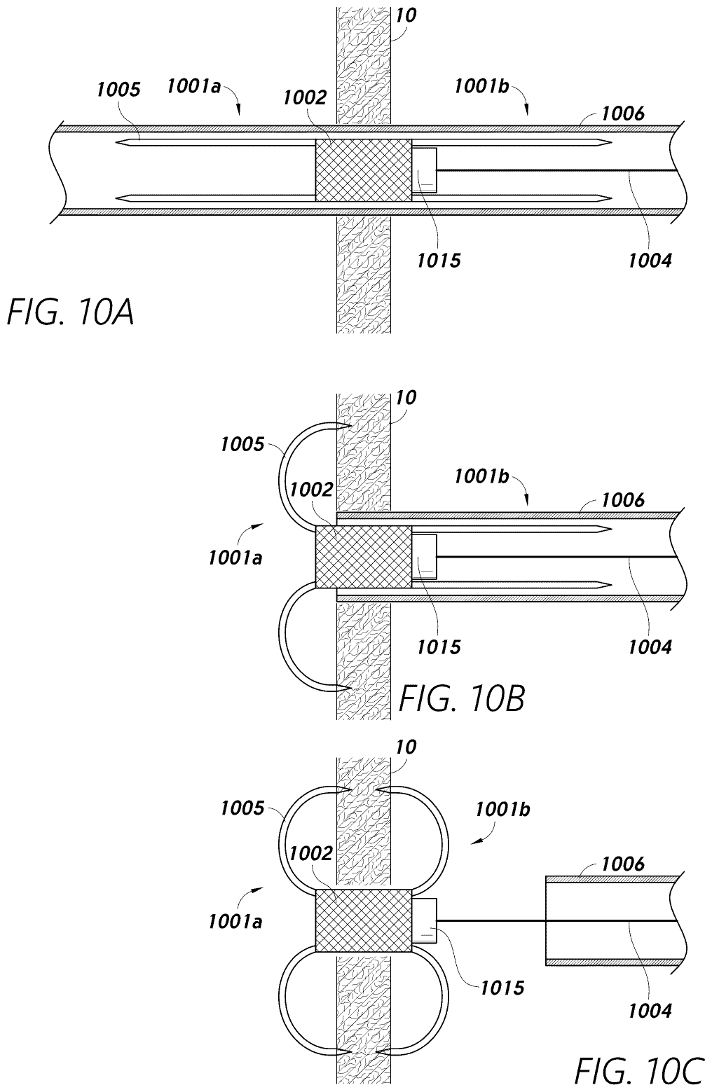

FIGS. 10A-10C illustrate delivery steps of another example shunt device comprising one or more expandable flanges 1001 (e.g., anchoring mechanisms) including one or more self-expanding and/or otherwise expandable anchoring arms 1005. In some examples, the shunt device may further comprise one or more shunt portions 1002 situated between a first flange 1001a and a second flange 1001b. The shunt portion 1002 and/or one or more flanges may be at least partially composed of one or more shape-memory alloys (e.g., Nitinol). The one or more anchoring arms 1005 may include any means for anchoring the shunt device to the tissue wall 10 and/or area of tissue within a body. The one or more flanges 1001 (e.g., including a distal flange 1001a and/or a proximal flange 1001b) may be configured to anchor to/into the tissue wall 10. While the distal flange 1001a and the proximal flange 1001b are each shown comprising a pair of two anchoring arms 1005, the shunt device may have any number of anchoring arms 1005. In some examples, the shunt device may comprise one or more anchoring arms 1005 at a first end of the shunt device and/or one or more anchoring arms 1005 at or near a second end of the shunt device. An anchoring arm 1005 may attach to and/or extend from any portion of the shunt device, including at least one or more cords forming the shunt portion 1002.

In some examples, the shunt portion(s) 1002 may be at least partially expandable. For example, the shunt portion(s) 1002 may comprise an interwoven structure and/or may be configured to expand and/or compress in length and/or width (e.g., similar to a finger trap) as necessary during delivery of the shunt device. For example, for situations in which a shunt device is delivered between blood vessels that are not adjacent, the shunt portion(s) 1002 may be configured to expand to extend across a gap between the blood vessels.

FIG. 10A illustrates a first delivery step in which the shunt device is delivered via a catheter 1006 and/or other delivery systems to through and/or at least partially within the tissue wall 10. FIG. 10B illustrates a second step of the delivery process in which the distal flange 1001a exits and/or is removed from the catheter 1006. Upon removal from the catheter 1006, the one or more distal anchoring arms 1005 may be configured to assume a default form in which the one or more distal anchoring arms 1005 may extend at least partially outward and/or may assume a retrograde deflection towards the proximal end of the shunt device and/or may form an approximately 90- to 180-degree angle with respect to the shunt device and/or may terminate in a hook pattern and/or form. In some examples, the one or more distal anchoring arms 1005 may be configured to be withdrawn slightly to capture the tissue wall 10.

FIG. 10C illustrates a third step of the delivery process in which the proximal flange 1001b may be deployed. For example, the catheter 1006 may be further retracted to at least partially expose the proximal anchoring arms 1005 and/or to otherwise cause the one or more proximal anchoring arms 1005 to assume a default form. The one or more proximal anchoring arms 1005 may not be attached to the one or more distal anchoring arms 1005. The one or more proximal anchoring arms 1005 (i.e., the proximal flange 1001b) may be configured deflect in an antegrade manner towards the distal anchoring arms 1005 (i.e., the distal flange 1001a).

In some examples, a pusher 1015 may be advanced to advance the proximal flange 1001b toward the tissue wall 10 to capture the tissue wall 10. Moreover, advancement of the pusher 1015 may be configured to cause compression of multiple blood vessels and/or tissue walls. While the anchoring arms 1005 of the flanges 1001 are shown penetrating the tissue wall 10, the flanges 1001 and/or anchoring arms 1005 may be configured to engage the tissue wall in any suitable manner. For example, the flanges 1001 may comprise one or more pedals and/or similar features configured to extend radially outward and/or away from the shunt portion 1002 during deployment and/or configured to contact the sides of the tissue wall 10 without penetrating the tissue wall 10. In some examples, a wire 1004 and/or similar device may be coupled to the pusher to apply force to the pusher and/or shunt implant.

Compression of the multiple blood vessels and/or tissue walls 10 may facilitate a hermetic seal between the blood vessels and/or tissue walls 10. In some examples, the proximal flange 1001b may be advanced until the one or more proximal anchoring arms 1005 engage over a proximal edge of the shunt portion 1002 and/or clip/lock into place. The anchoring arms 1005 may be configured to bend to an adjustable thickness of the tissue wall(s) 10 to maintain stability to the shunt device. In some examples, a diameter of a lumen through the shunt portion 1002 may be once locked by compressing the distal flange 1001a and the proximal flange 1001b together to cause deformation of the shunt portion 1002.

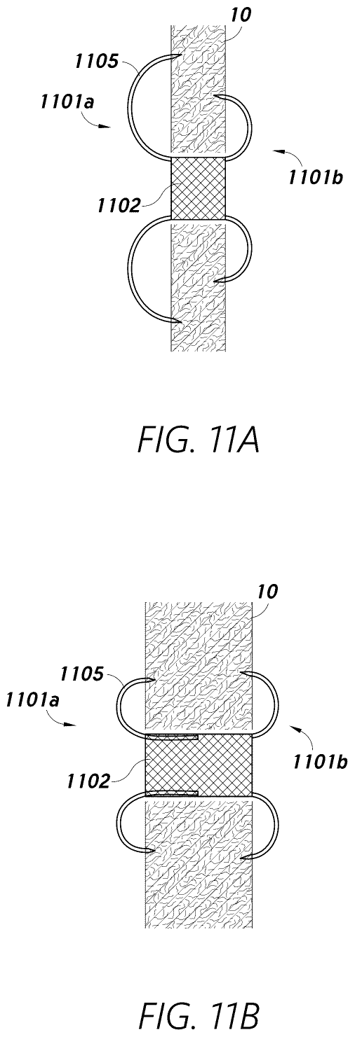

FIGS. 11A and 11B illustrate another example shunt device deployed at least partially within a tissue wall 10 in accordance with one or more examples. In some examples, the shunt device may comprise a shunt portion 1102 situated at least partially between a distal flange 110la comprising one or more distal anchoring arms 1005 and/or a proximal flange 1101b comprising one or more proximal anchoring arms 1105. The various anchoring arms 1105 may have asymmetric and/or different lengths. For example, the one or more distal anchoring arms 1105 may have a greater length than the one or more proximal anchoring arms 1105. The length of the one or more distal anchoring arms 1105 may be configured to facilitate adjustment of the shunt device to a size of the tissue wall 10. For example, as shown in FIG. 11A, for cases in which the tissue wall 10 has a relatively small width, the distal anchoring arms 1105 may be configured to extend a greater distance along a distal surface of the tissue wall 10. In another example, as shown in FIG. 11B, for cases in which the tissue wall 10 has a relatively large width, the distal anchoring arms 1105 may be configured to extend a greater length within an opening through the tissue wall and/or may extend a relatively small distance along the distal surface of the tissue wall 10.

The shunt device illustrated in FIGS. 11A and 11B may comprise asymmetric flanges 1101 and/or anchoring arms 1105. The distal flange 1101a and/or distal anchoring arms 1105 may be configured to be deployed after deployment of the proximal flange 1101b and/or proximal anchoring arms 1105. For example, the proximal flange 1101b (e.g., comprising relatively smaller anchoring arms 1105) may be deployed first. Following deployment of the proximal flange 1101b, the shunt device may be at least partially withdrawn to engage the proximal flange 1101b with the proximal side of the tissue wall 10. The distal flange 110la (e.g., comprising relatively larger anchoring arms 1105) may then be deployed to engage distal anchoring arms 1105 of the distal flange 1101a with the distal side of the tissue wall 10. The distal flange 1101a may comprise relatively long anchoring arms 1105 to allow the distal flange 1101a to engage the distal side of the tissue wall 10 without concern over the width of the tissue wall 10. For example, the distal flange 110la may be configured to self-adjust to varying tissue wall 10 widths based on the relative lengths of the anchoring arms 1105 of the distal flange 1101a. For wider tissue walls 10, the distal flange 1101a may extend a greater distance within the tissue wall 10 and/or may extend a smaller distance along the distal side of the tissue wall 10 while still forming a secure engagement with the distal side of the tissue wall. For thinner tissue walls 10, the distal flange 1101a may extend a smaller distance within the tissue wall 10 and/or may extend a greater distance along the distal side of the tissue wall 10.

In some examples, delivery of the shunt device may be performed such that the distal flange 1101a may be oriented toward a blood vessel providing relatively more room for the distal anchoring arms 1105 to expand. For example, the distal surface of the tissue wall 10 may be situated within a blood vessel having sufficient diameter for the relatively long distal anchoring arms 1105 to enter without piercing and/or contacting surfaces of the blood vessel other than the distal surface of the tissue wall 10.

For cases in which the tissue wall 10 has a relatively large width (as shown in FIG. 11B), the one or more distal anchoring arms 1105 may not extend a maximum amount beyond the distal surface of the tissue wall 10. In such cases, a portion of the distal anchoring arms 1105 may remain in an un-expanded and/or un-extended state and/or may be configured to extend generally linearly through the opening in the tissue wall. The one or more distal anchoring arms 1105 may, in such cases, contribute to the barrel length of the shunt device 1102. In some examples, the shunt device and/or distal anchoring arms 1105 may be self-adjusting and/or may adjust naturally to the size and/or thickness of the tissue wall 10.

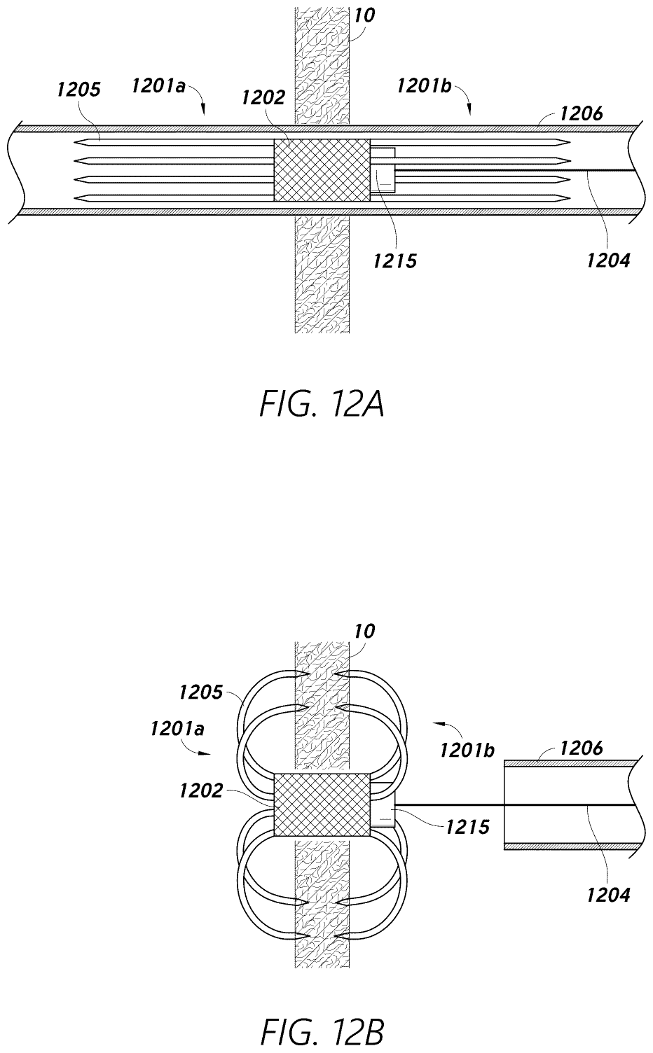

FIGS. 12A and 12B illustrate delivery steps of another example shunt device comprising one or more self-expanding and/or otherwise expandable flanges 1201, each comprising one or more anchoring arms 1205 (e.g., each comprising four anchoring arms 1205). In some examples, the shunt device may further comprise one or more shunt portions 1202 situated between sets of flanges 1201. The shunt portion 1202 and/or flanges 1201 may be at least partially composed of one or more shape-memory alloys (e.g., Nitinol). The one or more flanges 1201 may include any means for anchoring the shunt device to the tissue wall 10 and/or area of tissue within a body. The one or more flanges 1201 (e.g., including one or more distal anchoring arms 1205 and/or one or more proximal anchoring arms 1205) may be configured to anchor to/into the tissue wall 10. While the shunt device is shown comprising two flanges 1201 each including four anchoring arms 1205, the shunt device may have any number of anchoring arms 1205. In some examples, the shunt device may comprise a distal flange 1201a at a first end of the shunt device and/or a proximal flange 1201b at or near a second end of the shunt device. An anchoring arm 1205 may attach to and/or extend from any portion of the shunt device, including at one or more cords forming the shunt portion 1202. The shunt device may be delivered via one or more guidewires 1204 and/or catheters 1206. One or more stoppers 1215 may be disposed adjacent to the shunt portion 1202.

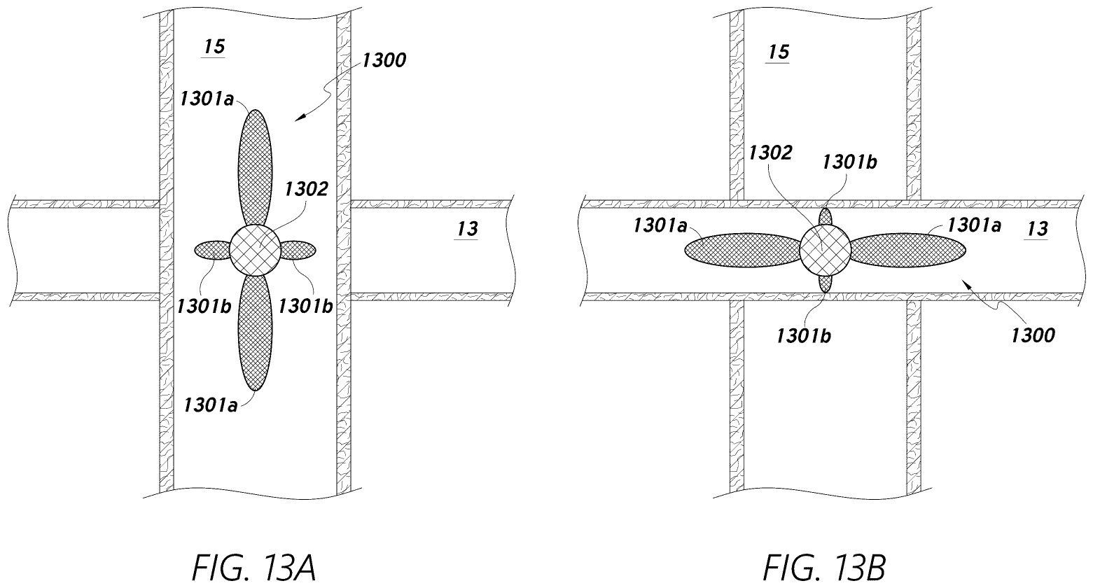

FIGS. 13A and 13B illustrate example shunt devices 1300 comprising one or more distal flanges 1301a configured to extend from a shunt portion 1302 and/or approximately perpendicularly from proximal flanges 1301b to facilitate delivery of the shunt devices 1300 across generally perpendicular blood vessels in accordance with one or more examples. For example, the shunt device 1300 may be configured to shunt blood between the RPA 13 and the SVC 15 and/or to accommodate the perpendicularity of the RPA-SVC orientation. In some cases, there may be potential for motion between the RPA 13 and the SVC 15 due at least in part to displacement of the right lung and/or SVC 15 during filling. Accordingly, the shunt device 1300 may comprise additional device features that can provide added force required to assure stabilization and/or approximation of the two RPA 13 and SVC 15. In some examples, the flanges 1301 that approximate the two vessels may be configured to extend in the long axis of each respective vessel to provide greater contact force while minimizing the stress imparted on the tissue that may otherwise be caused due to vessel deformation.

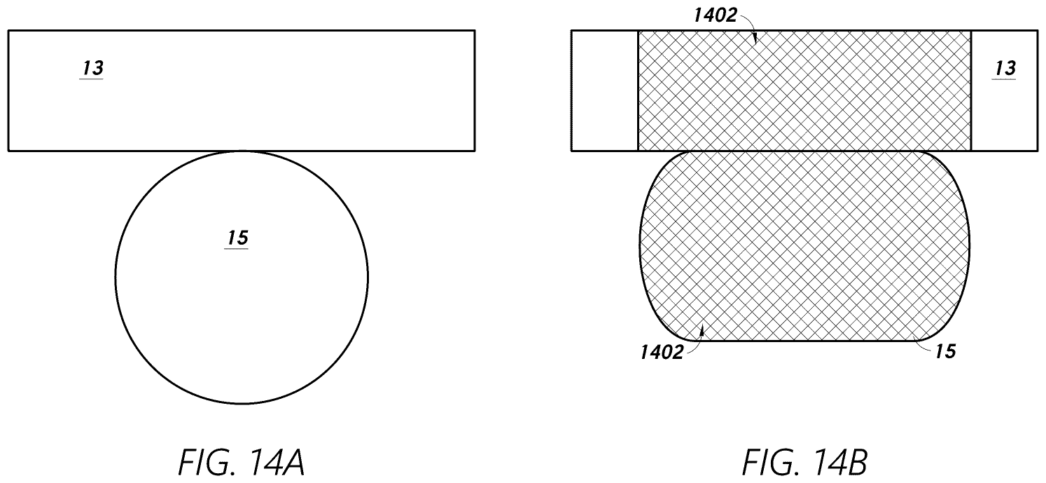

FIGS. 14A and 14B illustrate an intersection between a first blood vessel 13 and a second blood vessel 15. In some examples, one or more locking mechanisms 1402 may be utilized to control an amount of contact between the first blood vessel 13 and the second blood vessel 15 before, during, and/or after delivery of one or more shunt devices between the first blood vessel 13 and the second blood vessel 15. In some examples, for a single-and/or two-catheter system, one or more magnets and/or expandible balloons and/or shunt elements may be used as locking mechanisms 1402. The locking mechanisms 1402 may be configured to operate for each respective catheter or catheter section, such that the catheters or catheter locations are co-located. This approach can support seamless puncture and/or minimize risk of missing the puncture location. Either the magnetic sections or stents can force contact between the first blood vessel 13 and the second blood vessel 15 and/or maintain the positions of the first blood vessel 13 and the second blood vessel 15 during the puncture. A hybrid technique may also be used. For example, a shunt or balloon may contain one or more magnetic elements. In some examples, the shunt device may use radiopaque markers as a fluoroscopic indicator of puncture and/or delivery alignment. These approaches can help prevent the first blood vessel 13 and the second blood vessel 15 from moving away from each other during puncture or delivery system advancement.

In some examples, one or more tension wires may be utilized to enable acute right-angle flexion of the one or more catheters. This approach can enable straight access delivery of the shunt despite being within a perpendicular vascular system. Additional catheter features that can help facilitate ease of delivery can include flex wires for steering and/or a short nose cone, in addition to laser-cut stainless steel support members that can maintain pushability while promoting flexion and the angle of entry unique to the delivery of the device. Catheters and/or shunt devices can also comprise marker bands to improve ease of imaging. In some examples, the shunt device may be configured to deform to the left side of the heart if puncturing is performed from the SVC into the RPA towards the right ventricle.

The locking mechanism(s) 1402 may be used to approximate and/or stabilize the blood vessels during placement of one or more shunt devices and/or to facilitate alignment of the blood vessels for puncturing the one or more blood vessels. In some examples, the locking mechanism(s) 1402 can be part of one or more shunt device and/or may be a permanent implant that can be used to support long-term approximation and/or stability of the one or more shunt devices and/or blood vessels.

In some examples, the locking mechanism(s) 1402 may be coupled to and/or extend from one or more shunt devices. For example, a shunt device comprising the locking mechanisms(s) 1402 may be configured to lock and/or maintain apposition and/or to reduce risk of infiltration. In some examples, the shunt device and/or locking mechanism(s) 1402 may comprise one or more fluoroscopic markers for use in puncturing the blood vessels. Following puncturing of the blood vessels, the shunt device may be delivered and/or may be coupled to the locking mechanism(s) 1402.

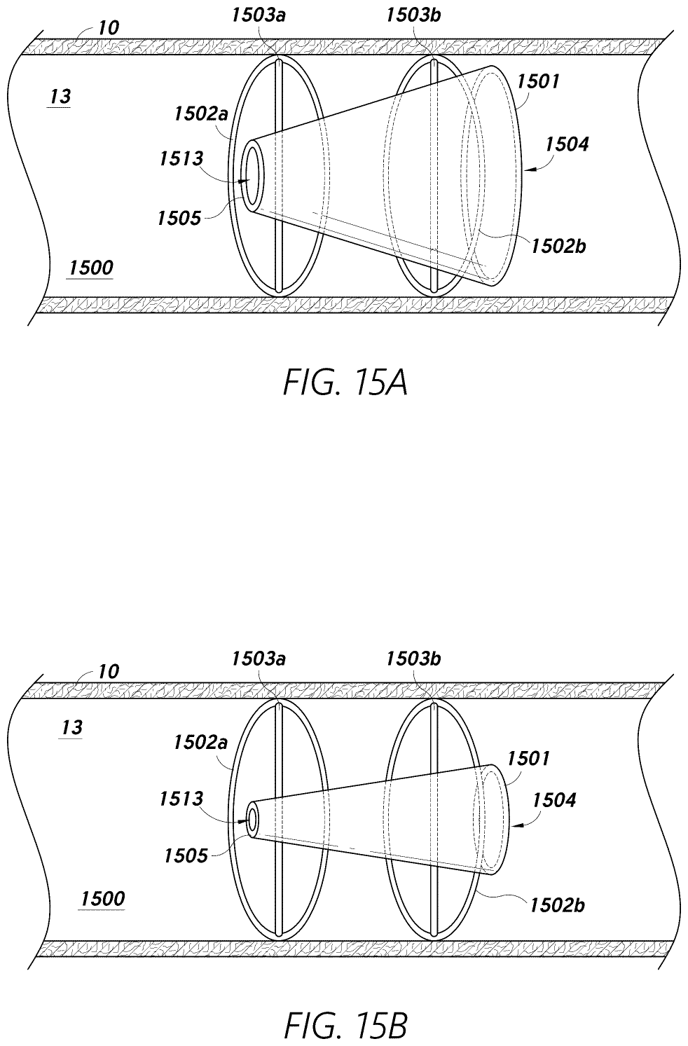

FIGS. 15A and 15B illustrate an example shunt device 1500 comprising a gasket and/or pressure-dependent orifice regulator that can modulate an amount of shunting to preferentially divert blood flow at higher pressures and minimize diversion at healthier pulmonary arterial pressures, in accordance with one or more examples. In some examples, the shunt device 1500 may be configured to minimize cell attachment under non-dynamic and/or baseline conditions. For example, the shunt device 1500 may comprise a pressure regulating system that can be isolated from vessel walls 10, thereby minimizing migration of cells (e.g., myofibroblasts). In some examples, the shunt device 1500 geometry may promote fluid shear across both the vessel wall 10 and shunt structure to minimize cell attachment from the circulating cells (e.g., fibroblasts).

FIG. 15A illustrates the shunt device 1500 in a default cross-section and/or in normal conditions. FIG. 15B illustrates the shunt device 1500 in an at least partially deformed condition and/or in response to an increased pressure gradient across the shunt device 1500. Deformation of the shunt device 1500 may be at least partially enabled by a “sail” like shunt structure 1501 attached to one or more frames 1502 which may be at least partially composed of one or more memory deformable materials (e.g., Nitinol). The shunt structure 1501 may have a conical and/or “funnel” shape in which a proximal end portion 1505 may have a smaller diameter and/or width than a distal end portion 1504. While the shunt structure 1501 is shown having a generally circular cross section, the shunt structure 1501 may have any suitable cross-sectional shape, including varying conical, triangular, hexagonal, pentagonal, and/or octagonal configurations. In some examples, the shunt structure 1501 may be configured to have an appropriate angle and/or percent of luminal cross-sectional area such that greater force is exerted under conditions of, for example, exercise or acute increases in pulmonary pressure that augment the pressure gradient to the SVC. Accordingly, the shunt device 1500 may be adaptive to changes in pressure and/or flow. In some examples, the shunt structure 1501 may have a suitable angle to promote fluid shear across the surface of the shunt structure 1501, thereby minimizing the potential for circulating cell attachment. The shunt structure 1501 may be configured to at least partially enclose a lumen 1513 extending through the shunt structure 1501.

In some examples, at least a portion of the circumferential area of the shunt structure 1501 may not be in contact with the vessel walls 10, thereby minimizing the chance of cell migration and adhesion. The one or more frames 1502 may be configured to contact the vessel walls 10 and/or an interior surface of a vessel wall 10 while allowing the shunt structure 1501 to be at least partially displaced from the vessel walls 10. In this way, the shunt device 1500 may experience a reduction in pressure at rest, which may be further reduced during elevations in the pulmonary-venous pressure gradient (e.g., in the deformed configuration of FIG. 15B). In some examples, the shunt device 1500 may comprise an opening 1513 at an inflow portion of the shunt structure 1501 apex which can promote high fluid velocity and/or shear onto the endoluminal surface to prevent attachment and/or preserve device mobility.

Multiple frames 1502 may be utilized to anchor the shunt structure 1501 within the vessel 13. For example, a proximal frame 1502a may be configured to attach to the shunt structure 1501 at or near the proximal end 1505 of the shunt structure 1501 and/or a distal frame 1502b may be configured to attach to the shunt structure 1501 at or near the distal end 1504 of the shunt structure 1501. The one or more frames 1502 may comprise crossbars 1503 (e.g., a proximal crossbar 1503a and/or a distal crossbar 1503b) configured to bisect the one or more frames 1502 and/or extend at least partially through the shunt structure 1501. In some examples, the one or more frames 1502 may have generally circular forms and/or may be configured to approximate a curvature of the interior surface of the blood vessel 13.

In some examples, a delivery process for delivering the shunt device 1500 may involve delivery through multiple blood vessels. For example, the internal jugular vein and the right femoral vein would be used for dual access. The shunt device 1500 may be placed at the end of a transcatheter delivery system which can traverse the right atrium, right ventricle, into the right pulmonary artery. This delivery catheter may or may not have an end hole or side hole catheter to inject contrast to confirm location. The catheter, in some examples, would have one or more articulation points at the distal end of the catheter to allow for manipulation and angulation, with a needle at the distal end for puncture. In some examples, there may be a loop or a snare marker in the SVC to allow for targeted puncture, and/or capture of the distal end or wire as needed. The shunt device 1500 may be extended across the RPA-SVC to be placed and create a shunt between the RPA 13 to SVC.

In another example, a coiled wire, snare, or wire marker may be used to traverse the right atrium, right ventricle, and right pulmonary artery, with imaging guidance. The imaging guidance may come in the form of a catheter with end or side hole contrast angiography, with a radio-opaque tip, marker bands, or an echogenic tip, or a combination of the above. This marker can mark the RPA 13 site. The delivery catheter can then be utilized in the SVC from either the femoral vein or the internal jugular vein with one or more articulation points at the distal end of the delivery catheter to better facilitate targeted puncture of the SVC-RPA. With adjunct imaging guidance, the SVC and RPA may be punctured with subsequent placement of the device and creation of the shunt.

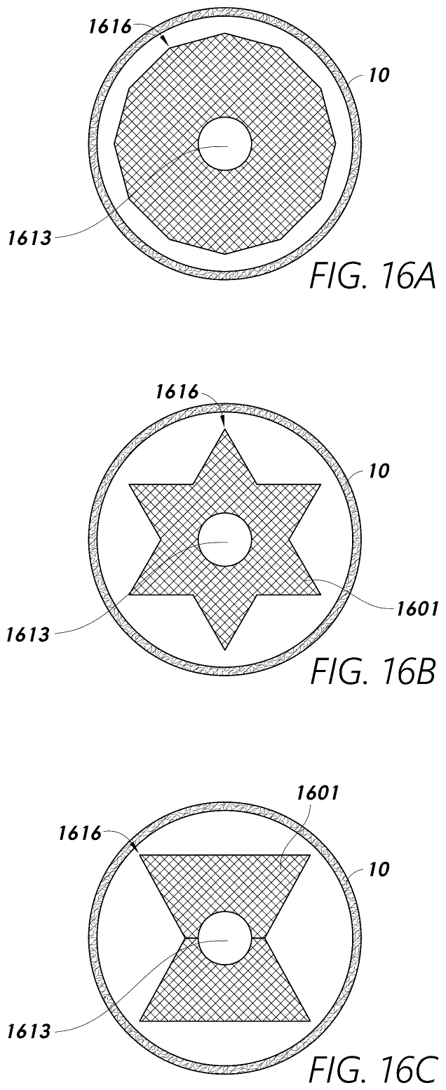

FIGS. 16A-16C illustrate cross-sectional views of example deformable shunt devices, in accordance with one or more examples. FIG. 16A illustrates an example default cross-sectional shape of the shunt device and FIGS. 16B and 16C illustrate deformed cross-sectional shapes of the shunt device. The shunt device may be configured to deform in response to changes in pressure and/or blood flow. The shunt device may be configured to deform and/or form multiple points 1616 around an outer diameter of the shunt device. Each point 1616 may comprise a spline (e.g., composed at least partially of Nitinol and/or other shape memory alloys), similar to an umbrella. The shunt device may not be directly attached to the vessel wall 10 and/or may be free-floating and/or de-coupled from the vessel wall 10.

The shunt device may comprise an opening 1613 at or near a middle portion of the shunt device. The opening 1613 may be configured to promote fluid shear though the center of the shunt device to prevent blood stagnation around the shunt device. In some examples, the shunt device may be anchored using one or more frames. The shunt device may comprise some generally rigid portions which may not be configured to flex. For example, the shunt device may comprise a distal portion (e.g., a nose portion) having a generally rigid form. In some examples, the shunt device may be configured to anchor and/or couple to a frame at one or more of the points 1616. The shunt device may comprise a sheet 1601 and/or occlusion element configured to extend around the opening 1613 and/or configured to form various shapes (e.g., a star-shaped form in FIG. 16B and/or a truncated cone shape in FIG. 16C).

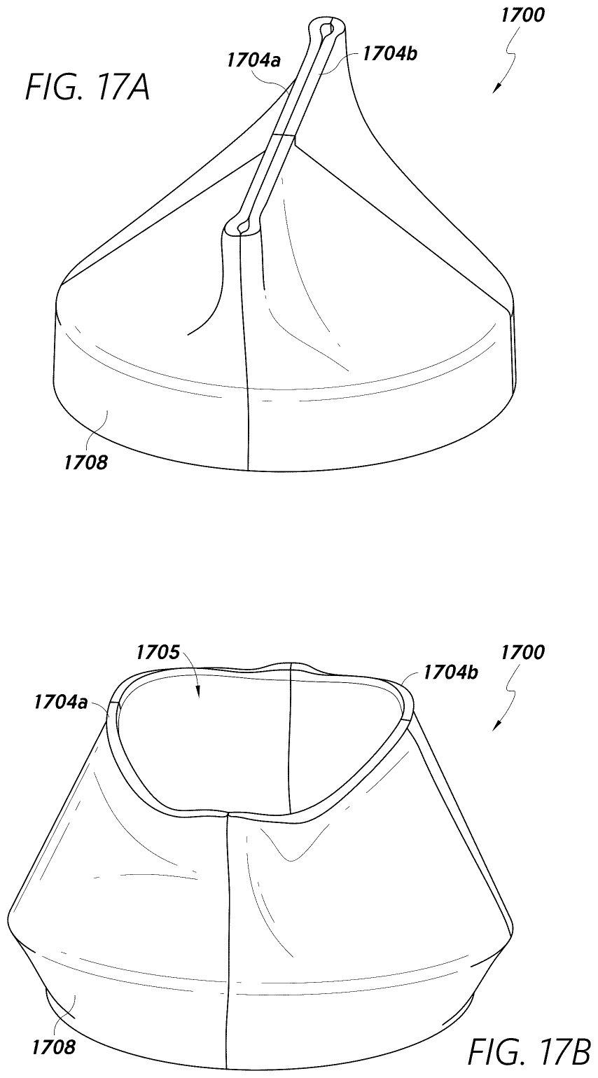

FIGS. 17A and 17B illustrate an example shunt device 1700 which may be configured for placement at a junction and/or intersection between two blood vessels in accordance with one or more examples. In some examples, the shunt device 1700 may be placed at the RPA/SVC junction to effectively reduce systolic and/or mean pulmonary artery pressure in patients while minimizing the impact on right ventricle function. While the pulmonary artery and SVC are described herein for illustration, the device may be a variable-orifice device.

The shunt device 1700 can comprise a valve shunt constructed of various metallic alloys and/or plastics. In some examples, the shunt device 1700 can include commissure posts configured to support the attachment of tissue leaflets 1704. The shunt device 1700 can be preloaded to a certain force level while the leaflet assembly may be pre-formed such that a first leaflet 1704a and/or second leaflet 1704b are naturally closed (as shown in FIG. 17A) under a zero-pressure gradient and/or a small defined pressure gradient. The leaflets 1704 may be configured to extend from a base 1708 of the shunt device 1700 and/or may have a generally tapered form.

The leaflets 1704 may be hydraulically loaded to deform the shunt device 1700 such that the leaflets 1704 may be open (as shown in FIG. 17B) to form an open orifice 1705 that allows blood flow. The leaflets 1704 can be configured to close when the pressure gradient drops below the set pressure or zero.

The shunt device 1700 may be configured to remain closed and/or to have a small opening 1705 at low pressure gradients (e.g., less than 20 mmHg) and/or during early right ventricle systole. At the mid-systole of the right ventricle when the pulmonary arterial pressure increases (e.g., exceeds 20 mmHg), the shunt device 1700 may be configured to deform and/or allow the valve leaflets 1704 to open and/or create a shunt flow (e.g., from the pulmonary artery to the SVC).

The shunt device 1700 can then reduce the orifice 1705 dimension (return to the closed position) as soon as the pulmonary artery pressure drops below a given pressure value (e.g., 20 mmHg). In this fashion, the pulmonary artery pressure can rise and fall in response to right ventricle contraction and relaxation but can remain within a small range of the set pressure (e.g., 20 mmHg). In this way, right ventricle overload may be avoided.

The set pressure of 20 mmHg is used as an example for illustrative purposes. However, this pressure can be adjusted through different design variation. The shunt can be customized to meet specific patient needs.

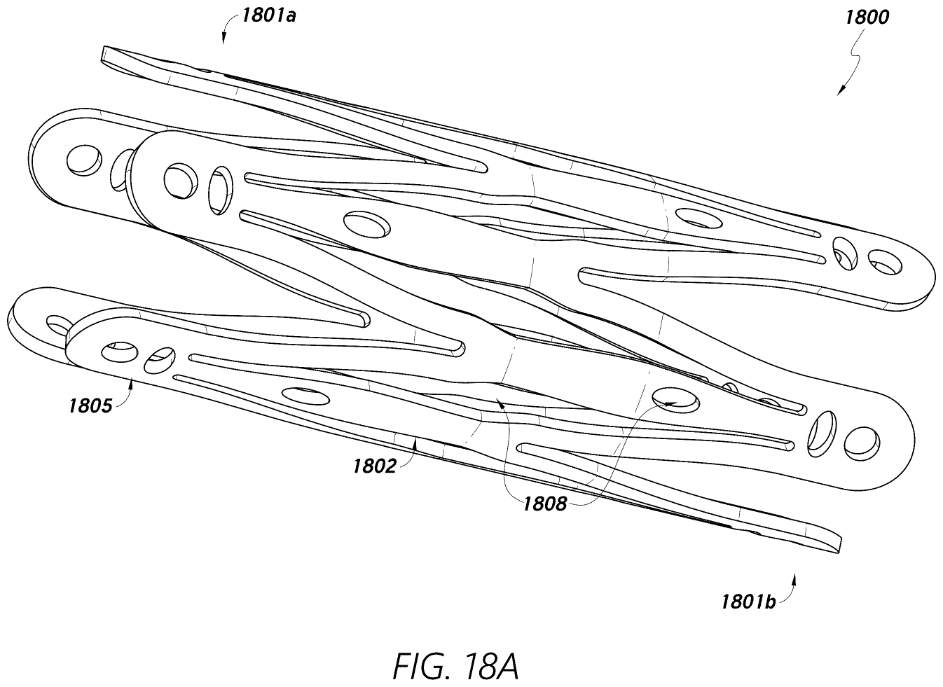

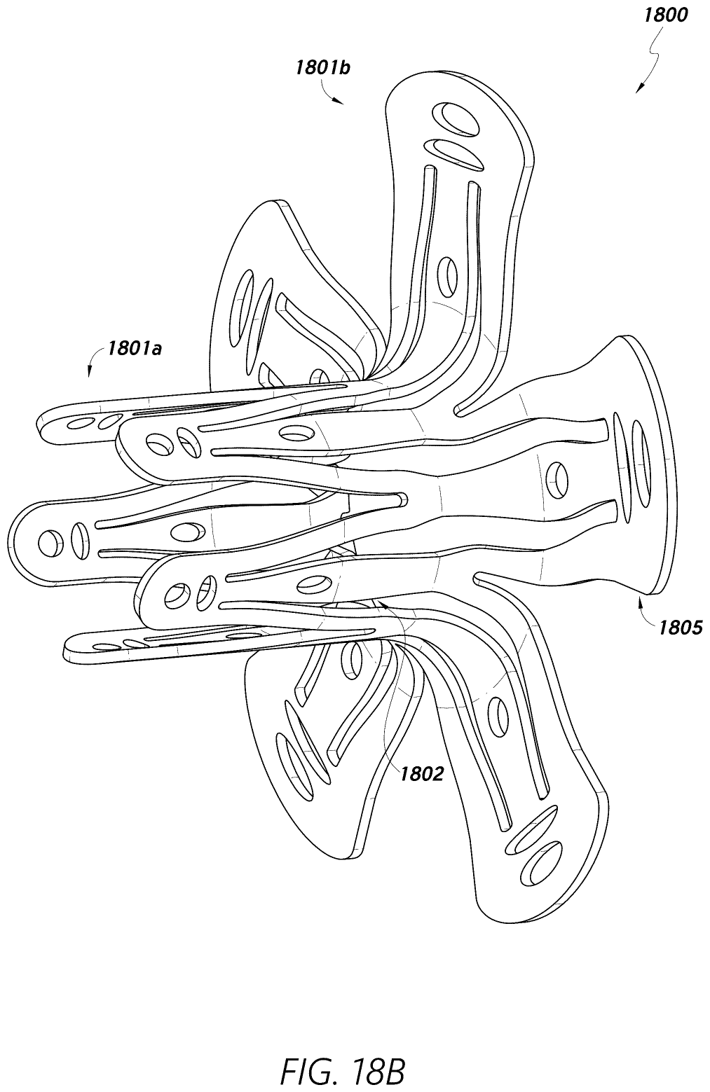

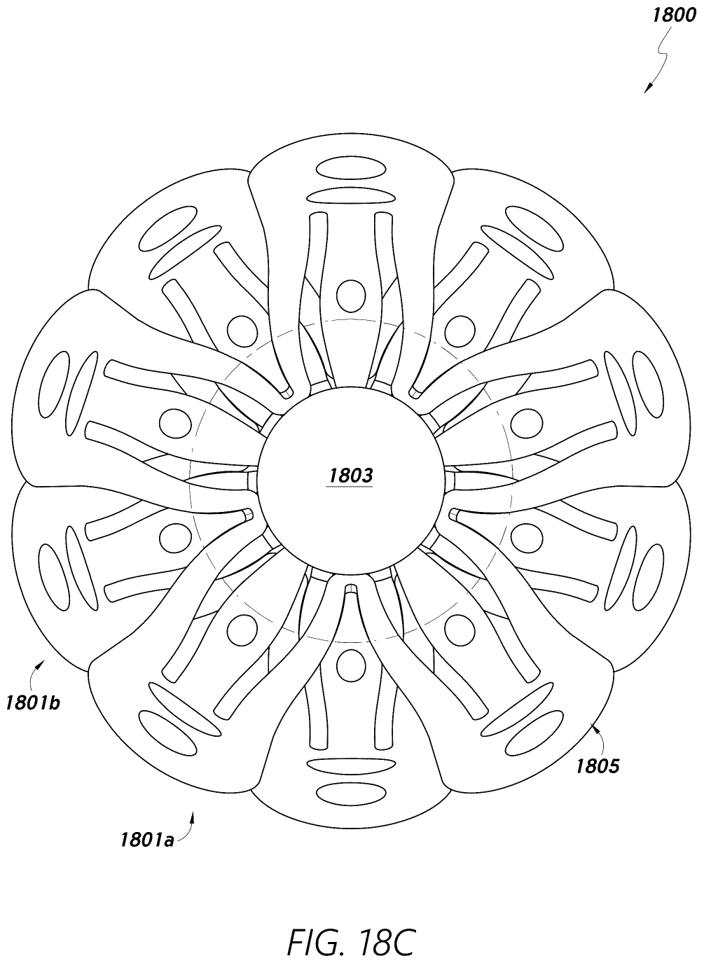

FIGS. 18A-18C illustrate another example shunt implant 1800 configured to shunt blood between blood flow pathways and/or cardiac chambers in accordance with one or more examples. The implant 1800 may comprise a shunt portion 1802 situated between a first anchoring mechanism 1801a (e.g., flange) and/or a second anchoring mechanism 1801b (e.g., flange). The implant 1800 may be configured for deployment between two vessel walls and/or may be configured to shunt blood from a first blood vessel to a second blood vessel. In some examples, the implant 1800 may be a star-shaped and/or flower-shaped device in which the anchoring mechanisms 1801 comprise petals 1805 (e.g., anchoring arms) configured to extend (following deployment) generally perpendicularly from the shunt portion 1802. While each anchoring mechanism 1801 is shown comprising five petals 1805, the implant 1800 may comprise any number and/or size of petals 1805 and/or similar anchoring features. The implant 1800 may comprise an inner lumen 1803 extending through the implant 1800.

The petals 1805 and/or shunt portion 1802 may comprise a network of inter-connected lengths of material (e.g., Nitinol and/or other shape-memory alloys) having apertures 1808 and/or gaps between the lengths of material. In some examples, the apertures 1808 may represent removed portions from a solid length of material.

FIG. 18A provides a side view of the implant 1800 in an undeployed state in which the petals 1805 may extend generally in parallel with the shunt portion 1802. In the undeployed state, an angle of extension between the shunt portion 1802 and the petals 1805 may be approximately zero degrees. The implant 1800 may be configured to assume the undeployed state during delivery via a delivery device (e.g., a catheter) to minimize a profile of the implant 1800 and/or of the delivery device.

FIG. 18B provides a side view of the implant 1800 in a partially deployed state in which the first anchoring mechanism 1801a is undeployed while the second anchoring mechanism 1801b is deployed. In some examples, the first anchoring mechanism 1801a and/or second mechanism 1801b may be configured to deploy independently. For example, if the first anchoring mechanism 1801a is still at least partially within a delivery device while the second anchoring mechanism 1801b is deployed beyond a distal end of the delivery device, the second anchoring mechanism 1801b may be configured to naturally deploy and/or petals 1805 of the second anchoring mechanism 1801b may be configured to assume a deployed state. In the deployed state, the petals 1805 may be configured to angle of extension of approximately seventy-five to one-hundred and forty-five degrees with respect to the shunt portion 1802. The angle of extension between the petals 1805 and the shunt portion (e.g., cylindrical and/or tubular body) can vary due to a bendability and/or elasticity of the petals 1805 and/or shunt portion 1802. FIG. 18C provides a frontal view of the implant 1800 in a fully deployed state in which the first anchoring mechanism 1801a and the second anchoring mechanism 1801b may be in deployed state. In some examples, the implant 1800 may be shape-set in the fully deployed form and/or may assume the undeployed form of FIG. 18A in response to external force from a delivery device.

In some examples, deployment of the petals 1805 may allow the implant 1800 to grasp and/or hook onto one or more vessel walls. For example, after the implant 1800 is extend through an opening of a vessel wall, the petals 1805 may be configured to extend at an angle from the shunt portion 1802 such that the petals 1805 grasp onto the vessel wall. The second anchoring mechanism 1801b may be configured to grasp onto a distal vessel wall and/or the first anchoring mechanism 1801a may be configured to grasp onto a proximal vessel wall. The second anchoring mechanism 1801b may be deployed first and/or the implant 1800 may then be retracted partially to allow the first anchoring mechanism 1801a to grasp onto the proximal vessel wall. When both anchoring mechanisms 1801 are deployed, the implant 1800 may be configured to compress a distal vessel and/or a proximal vessel together.

The implant 1800 may be configured to accommodate an angle of orientation between a first blood vessel (e.g., the RPA) and a second blood vessel (e.g., the SVC). For example, the RPA and SVC may generally have a perpendicular alignment. Accordingly, the petals 1805 may be configured to assume a generally perpendicular alignment with respect to the shunt portion 1802. The anchoring mechanisms 1801 may be configured to be extended to embody the respective vessels that the anchoring mechanisms 1801 anchor to. As a result, greater contact force may be achieved while minimizing stress imparted on the tissue.

Because of the potential for motion between the two blood vessels the implant 1800 is anchored to (e.g., due to displacement of the right lung and/or SVC during filling), the size of the shunt portion 1802 can be adjustable. In some examples, the implant 1800 may comprise various features configured to provide added force to ensure stabilization and/or approximation between the two blood vessels and/or vessel walls.

In some examples, various mechanisms may be threaded through one or more apertures 1808 of the implant 1800. For example, one or more pin mechanisms and/or threads may be threaded through the apertures to facilitate a secure hold between the two blood vessels. In some examples, tissue glue may be used to secure approximation of the blood vessels.

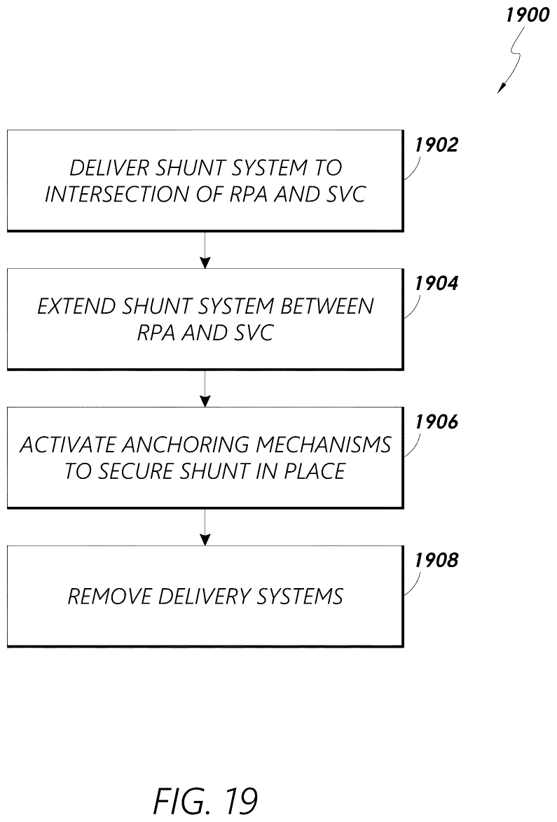

FIG. 19 illustrates a flowchart related to an example process 1900 for delivering one or more shunt implants to target locations within a body, which can include an intersection area between the RPA and the SVC, in accordance with one or more examples. At step 1902, the process 1900 involves delivering a shunt system, which can include one or more shunt devices and/or related delivery systems, via one or more blood vessels to a target location within a human patient (e.g., at or near an intersection point between the RPA and SVC). In some examples, the one or more shunt devices may be configured for percutaneous delivery. The delivery systems can include one or more catheters, guidewires, nose cones, stoppers, dilators, support sheaths and/or rods, dilation balloons, and/or other delivery devices.

At step 1904, the process 1900 involves extending at least a portion of the shunt system through a blood vessel wall of a first blood vessel (e.g., the RPA or SVC) until at least a portion of the shunt system (e.g., at least a portion of a distal flange of the shunt device) enters a second blood vessel (e.g., the RPA or SVC). At least a portion of the shunt device (e.g., a proximal flange) may remain at least partially situated within the first blood vessel. Moreover, at least a portion of the shunt device (e.g., a shunt portion) may remain at least partially embedded within a first vessel wall of the first blood vessel, a second vessel wall of the second blood vessel, and/or in an area between the first vessel wall and the second vessel wall.

In some examples, the shunt system may be at least partially balloon expandable. For example, the shunt system may comprise a balloon expandable shunt (e.g., at least partially composed of stainless steel) that can be expanded during a delivery procedure to further optimize a desired pressure drop and/or can be expanded further during subsequent procedures to increase therapeutic benefits.

At step 1906, the process 1900 involves activating one or more anchoring mechanisms (e.g., the distal flange and/or the proximal flange) to secure the shunt device and/or the shunt portion in place. In some examples, the distal flange and/or the proximal flange may be at least partially self-expanding. For example, the distal flange and/or the proximal flange may comprise one or more anchoring arms configured to extend at an angle from the shunt portion and/or to engage (e.g., pierce and/or otherwise contact) one or more vessel walls. In some examples, activating the distal flange and/or proximal flange may involve advancing one or more stoppers to at least partially compress and/or widen the distal flange and/or proximal flange to more securely anchor the shunt device and/or plug openings in the first vessel wall and/or second vessel wall.

At step 1908, the process 1900 involves removing the delivery systems while the shunt device remains anchored in the first blood vessel and/or second blood vessel.

Some implementations of the present disclosure relate to a shunt device comprising a first shunt portion configured to maintain a blood flow pathway between a first blood vessel and a second blood vessel, a first proximal flange configured to anchor the first shunt portion in place and configured for placement within the first blood vessel, and a first distal flange configured to anchor the first shunt portion in place. The first shunt portion is situated at least partially between the first proximal flange and the first distal flange.

In some examples, the first proximal flange and the first distal flange are configured to compress lengthwise and expand in width following deployment. The first shunt portion may extend from the first proximal flange and the first distal flange.

The first proximal flange may comprise multiple self-expanding arms configured to extend generally perpendicularly from the first shunt portion. In some examples, the first shunt portion may have an adjustable length.

In some examples, the first distal flange is configured for placement within the second blood vessel. The first blood vessel and the second blood vessel may have generally perpendicular orientations with respect to each other.

The shunt device may further comprise a second shunt portion configured to maintain the blood flow pathway between the first blood vessel and the second blood vessel, a second proximal flange configured to anchor the first shunt portion in place and configured for placement between the first blood vessel and the second blood vessel, and a second distal flange configured to anchor the first shunt portion in place and configured for placement within the second blood vessel. In some examples, the first shunt portion is situated at least partially between the first proximal flange and the first distal flange.

The first distal flange may be configured for placement between the first blood vessel and the second blood vessel. In some examples, shunt device further comprises a third shunt portion situated between the first distal flange and the second proximal flange.

In some examples, the first proximal flange comprises multiple anchoring arms configured to assume a retrograde deflection towards the first distal flange to anchor to a proximal side of a tissue wall.

The first distal flange may comprise multiple anchoring arms configured to assume a retrograde deflection towards the first proximal flange to anchor to a distal side of the tissue wall. In some examples, the anchoring arms of the first proximal flange have greater lengths than the anchoring arms of the first distal flange.

In accordance with some implementations of the present disclosure, a method comprises percutaneously delivering a shunt device into a first blood vessel near an intersection point between the first blood vessel and a second blood vessel. The shunt device comprises a shunt portion, a proximal flange, and a distal flange. The method further comprises passing the shunt device at least partially through a vessel wall of the first blood vessel until the distal flange enters the second blood vessel.

The method may further comprise twisting a threaded wire member to compress the proximal flange lengthwise. In some examples, the method further comprises crimping the shunt portion, proximal flange, and distal flange to a fixed portion extending through the shunt portion, proximal flange, and distal flange.

In some examples, the shunt device is situated at least partially between a distal stopper and a proximal stopper. The method may further comprise reducing a distance between the distal stopper and the proximal stopper to cause lengthwise compression of the proximal flange and the distal flange.

Some implementations of the present disclosure relate to a shunt device comprising a shunt portion configured to shunt blood through a blood vessel. The shunt portion has a first end and a second end. The first end has a smaller width than the second end. The shunt device further comprises a first anchoring mechanism coupled to the shunt portion near the first end and a second anchoring mechanism coupled to the shunt portion near the second end.

The first anchoring mechanism may further comprise a generally circular frame configured to extend around an interior surface of the blood vessel. In some examples, the first anchoring mechanism comprises a crossbar extending from the frame and configured to extend through the shunt portion.

In some examples, a cross section of the second end may be configured to deform in response to changes in blood flow in the blood vessel. The second end may be configured to open and close in response to pressure changes in the blood vessel.

### Additional Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A shunt device comprising a first shunt portion configured to maintain a blood flow pathway between a first blood vessel and a second blood vessel, a first proximal flange configured to anchor the first shunt portion in place and configured for placement within the first blood vessel, and a first distal flange configured to anchor the first shunt portion in place, wherein the first shunt portion is situated at least partially between the first proximal flange and the first distal flange.

Example 2: The shunt device of any example herein, in particular example 1, wherein the first proximal flange and the first distal flange are configured to compress lengthwise and expand in width following deployment.

Example 3: The shunt device of any example herein, in particular example 1, wherein the first shunt portion extends from the first proximal flange and the first distal flange.

Example 4: The shunt device of any example herein, in particular example 1, wherein the first proximal flange comprises multiple self-expanding arms configured to extend generally perpendicularly from the first shunt portion.

Example 5: The shunt device of any example herein, in particular example 1, wherein the first shunt portion may have an adjustable length.

Example 6: The shunt device of any example herein, in particular example 1, wherein the first distal flange is configured for placement within the second blood vessel, and wherein the first blood vessel and the second blood vessel have generally perpendicular orientations with respect to each other.

Example 7: The shunt device of any example herein, in particular example 1, further comprising a second shunt portion configured to maintain the blood flow pathway between the first blood vessel and the second blood vessel, a second proximal flange configured to anchor the first shunt portion in place and configured for placement between the first blood vessel and the second blood vessel, and a second distal flange configured to anchor the first shunt portion in place and configured for placement within the second blood vessel, wherein the first shunt portion is situated at least partially between the first proximal flange and the first distal flange, and wherein the first distal flange is configured for placement between the first blood vessel and the second blood vessel.

Example 8: The shunt device of any example herein, in particular example 7, further comprising a third shunt portion situated between the first distal flange and the second proximal flange.

Example 9: The shunt device of any example herein, in particular example 1-8, wherein the first proximal flange comprises multiple anchoring arms configured to assume a retrograde deflection towards the first distal flange to anchor to a proximal side of a tissue wall.

Example 10: The shunt device of any example herein, in particular example 9, wherein the first distal flange comprises multiple anchoring arms configured to assume a retrograde deflection towards the first proximal flange to anchor to a distal side of the tissue wall.

Example 11: The shunt device of any example herein, in particular example 10, wherein the anchoring arms of the first proximal flange have greater lengths than the anchoring arms of the first distal flange.

Example 12: A method comprising percutaneously delivering a shunt device into a first blood vessel near an intersection point between the first blood vessel and a second blood vessel, the shunt device comprising a shunt portion, a proximal flange; and a distal flange, and passing the shunt device at least partially through a vessel wall of the first blood vessel until the distal flange enters the second blood vessel.

Example 13: The method of any example herein, in particular example 12, further comprising twisting a threaded wire member to compress the proximal flange lengthwise.

Example 14: The method of any example herein, in particular example 12, further comprising crimping the shunt portion, proximal flange, and distal flange to a fixed portion extending through the shunt portion, proximal flange, and distal flange.

Example 15: The method of any example herein, in particular example 12, wherein the shunt device is situated at least partially between a distal stopper and a proximal stopper, and the method further comprises reducing a distance between the distal stopper and the proximal stopper to cause lengthwise compression of the proximal flange and the distal flange.

Example 16: A shunt device comprising a shunt portion configured to shunt blood through a blood vessel, the shunt portion having a first end and a second end, wherein the first end has a smaller width than the second end, a first anchoring mechanism coupled to the shunt portion near the first end, and a second anchoring mechanism coupled to the shunt portion near the second end.

Example 17: The shunt device of any example herein, in particular example 16, wherein the first anchoring mechanism further comprises a generally circular frame configured to extend around an interior surface of the blood vessel.

Example 18: The shunt device of any example herein, in particular example 17, wherein the first anchoring mechanism comprises a crossbar extending from the frame and configured to extend through the shunt portion.

Example 19: The shunt device of any of any example herein, in particular example 16, wherein a cross section of the second end is configured to deform in response to changes in blood flow in the blood vessel.

Example 20: The shunt device of any example herein, in particular example 16, wherein the second end is configured to open and close in response to pressure changes in the blood vessel.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A shunt device comprising:
a first shunt portion configured to maintain a blood flow pathway between a first blood vessel and a second blood vessel;
a first proximal flange configured to anchor the first shunt portion in place and configured for placement within the first blood vessel; and
a first distal flange configured to anchor the first shunt portion in place;
wherein the first shunt portion is situated at least partially between the first proximal flange and the first distal flange.

2. The shunt device of claim 1, wherein the first proximal flange and the first distal flange are configured to compress lengthwise and expand in width following deployment.

3. The shunt device of claim 1, wherein the first shunt portion extends from the first proximal flange and the first distal flange.

4. The shunt device of claim 1, wherein the first proximal flange comprises multiple self-expanding arms configured to extend generally perpendicularly from the first shunt portion.

5. The shunt device of claim 1, wherein the first shunt portion may have an adjustable length

6. The shunt device of claim 1, wherein the first distal flange is configured for placement within the second blood vessel, and wherein the first blood vessel and the second blood vessel have generally perpendicular orientations with respect to each other.

7. The shunt device of claim 1, further comprising:
a second shunt portion configured to maintain the blood flow pathway between the first blood vessel and the second blood vessel;
a second proximal flange configured to anchor the first shunt portion in place and configured for placement between the first blood vessel and the second blood vessel; and
a second distal flange configured to anchor the first shunt portion in place and configured for placement within the second blood vessel;
wherein the first shunt portion is situated at least partially between the first proximal flange and the first distal flange, and wherein the first distal flange is configured for placement between the first blood vessel and the second blood vessel.

8. The shunt device of claim 7, further comprising a third shunt portion situated between the first distal flange and the second proximal flange.

9. The shunt device of claim 1, wherein the first proximal flange comprises multiple anchoring arms configured to assume a retrograde deflection towards the first distal flange to anchor to a proximal side of a tissue wall.

10. The shunt device of claim 9, wherein the first distal flange comprises multiple anchoring arms configured to assume a retrograde deflection towards the first proximal flange to anchor to a distal side of the tissue wall.

11. The shunt device of claim 10, wherein the anchoring arms of the first proximal flange have greater lengths than the anchoring arms of the first distal flange.

12. A method comprising:
percutaneously delivering a shunt device into a first blood vessel near an intersection point between the first blood vessel and a second blood vessel, the shunt device comprising:
a shunt portion;
a proximal flange; and
a distal flange; and
passing the shunt device at least partially through a vessel wall of the first blood vessel until the distal flange enters the second blood vessel.

13. The method of claim 12, further comprising twisting a threaded wire member to compress the proximal flange lengthwise.

14. The method of claim 12, further comprising crimping the shunt portion, proximal flange, and distal flange to a fixed portion extending through the shunt portion, proximal flange, and distal flange.

15. The method of claim 12, wherein the shunt device is situated at least partially between a distal stopper and a proximal stopper, and the method further comprises reducing a distance between the distal stopper and the proximal stopper to cause lengthwise compression of the proximal flange and the distal flange.

16. The method of claim 12, wherein the first blood vessel is a superior vena cava and the second blood vessel is a right pulmonary artery.

17. A shunt device comprising:
a shunt portion configured to shunt blood through a blood vessel, the shunt portion having a first end and a second end, wherein the first end has a smaller width than the second end;
a first anchoring mechanism coupled to the shunt portion near the first end; and
a second anchoring mechanism coupled to the shunt portion near the second end.

18. The shunt device of claim 17, wherein the first anchoring mechanism further comprises a generally circular frame configured to extend around an interior surface of the blood vessel.

19. The shunt device of claim 18, wherein the first anchoring mechanism comprises a crossbar extending from the frame and configured to extend through the shunt portion.

20. The shunt device of claim 17, wherein a cross section of the second end is configured to deform in response to changes in blood flow in the blood vessel.

21. The shunt device of claim 17, wherein the second end is configured to open and close in response to pressure changes in the blood vessel.
